(19) United States
(12) Patent Application Publication
(10) Pub. No.: US 2006/0094935 A1
(43) Pub. Date: May 4, 2006

(54) Portable psychophysiology system and method of use

(75) Inventors: Daniel M. Sussman, Flushing, NY (US); Paul Mazzucco, Schnecksville, PA (US); Matthew Gilvey, High Bridge, NJ (US); Michael A. Godsey, Whitehall, PA (US); John Willenbecher, Allentown, PA (US)

Correspondence Address: MICHAEL BEST & FRIEDRICH, LLP, 100 E WISCONSIN AVENUE, MILWAUKEE, WI, 53202, US

(73) Assignee: Coulbourn Instruments, L.L.C., Allentown, PA (US)

(21) Appl. No.: 11/255,065

(22) Filed: Oct. 20, 2005

Related U.S. Application Data
(60) Provisional application No. 60/620,655, filed on Oct. 20, 2004.

(51) Int. Cl. A61B 5/00; A61B 5/04

(52) U.S. Cl. 600/300; 600/544; 600/545; 600/546; 600/509; 128/905

(57) ABSTRACT

A portable biofeedback system including a test module and a personal computer for acquiring psychophysiological data. The test module includes a plurality of inputs for sensors for acquiring data from a patient. The test module also includes user manipulatable controls to adjust the hardware functionality of the test module. The personal computer includes a software program operable to receive and display the data from the test module. The software program allows the user to adjust the hardware functionality of the test module.

### RELATED APPLICATIONS

This application is a non-provisional application of and claims priority to U.S. Patent Application Ser. No. 60/620,655, filed on Oct. 20, 2004. The entire contents of U.S. Patent Application Ser. No. 60/620,655 are hereby incorporated by reference.

### BACKGROUND

It is typical for a patient to have to travel to a laboratory setting for tests. Many patients suffer from an increased stress level during the visit. As a result, the test results for many of the patients are affected due to the foreign, institutional laboratory surroundings.

### SUMMARY

It would be desirable to have a system that is portable and includes the same or similar functionality of the equipment used in the laboratory setting. The portable equipment can be transported to the patient's location, which is generally more relaxing and less stressful for the patient.

In one embodiment, the invention includes a biofeedback system comprising a test module and a processor. The test module includes an amplifier and filter circuit, an integrator circuit, a skin conductance circuit, and an audio signal generator circuit operable to output one of a tone signal and a white noise signal. The processor includes a software program operable to adjust functionality of the amplifier and filter circuit, the integrator circuit, and the audio signal generator circuit, an acquisition module operable to receive data from the test module, the data acquired from a patient, and a display module operable to display the received data.

In another embodiment, the invention includes a biofeedback system comprising a plurality of test modules and a processor. Each test module is configured to link to another test module and each test module includes an amplifier and filter circuit, and an integrator circuit. The processor is connected to one of the test modules, and the processor includes a software program operable to adjust functionality of the amplifier and filter circuit and the integrator circuit, an acquisition module operable to receive data from the plurality of test modules, the data acquired from a patient, and a display module operable to display the received data.

In yet another embodiment, the invention includes a method of acquiring psychophysiological data from a patient with a biofeedback system including a personal computer and a test module. The method comprises the act of the test module receiving instructions from the personal computer to adjust functionality of the test module, the test module delivering a plurality of stimuli to the patient, the test module acquiring data from the patient, the data reflective of a response of the patient to the delivered stimuli, transmitting the data to the personal computer, and displaying the data on the personal computer.

Other aspects of the invention will become apparent by consideration of the detailed description and accompanying drawings.

### BRIEF DESCRIPTION OF THE DRAWINGS

FIG. 1 illustrates a perspective view of a portable biofeedback system according to one embodiment of the invention.

FIG. 2 illustrates a perspective view of a test module of the portable biofeedback system of FIG. 1.

FIG. 3 illustrates a rear perspective view of the test module of FIG. 2.

FIG. 4 illustrates a schematic of the test module of FIG. 2.

FIGS. 5-8 illustrate a schematic diagram of a controller of the test module of FIG. 2.

FIGS. 9-11 illustrate a schematic diagram of one channel of an amplifier and filter circuit of the test module of FIG. 2.

FIGS. 12-13 illustrate a schematic diagram of one channel of an integrator circuit of the test module of FIG. 2.

FIGS. 14-16 illustrate a schematic diagram of a second channel of an amplifier and filter circuit of the test module of FIG. 2.

FIGS. 17-18 illustrate a schematic diagram of a second channel of an integrator circuit of the test module of FIG. 2.

FIGS. 19-21 illustrate a schematic diagram of a third channel of an amplifier and filter circuit of the test module of FIG. 2.

FIGS. 22-23 illustrate a schematic diagram of a third channel of an integrator circuit of the test module of FIG. 2.

FIGS. 24-26 illustrate a schematic diagram of a fourth channel of an amplifier and filter circuit of the test module of FIG. 2.

FIGS. 27-28 illustrate a schematic diagram of a fourth channel of an integrator circuit of the test module of FIG. 2.

FIGS. 29-30 illustrate circuit connections between the schematic diagrams of FIGS. 9-28.

FIGS. 31-33 illustrate a schematic diagram of a skin conductance circuit of the test module of FIG. 2.

FIGS. 34-37 illustrate a schematic diagram of an audio signal generator circuit of the test module of FIG. 2.

FIG. 38 illustrates a schematic of a personal computer of the portable biofeedback system of FIG. 1.

FIGS. 39-45 illustrate a plurality of user interface screens of a software program implemented in the personal computer of FIG. 38.

FIG. 46 illustrates a portable biofeedback system according to one embodiment of the invention.

FIG. 47 illustrates a method of the invention according to one embodiment of the invention.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, 37 connected” and “coupled” are not restricted to physical or mechanical connections or couplings.

In addition, it should be understood that embodiments of the invention include both hardware and electronic components or modules that, for purposes of discussion, may be illustrated and described as if the majority of the components were implemented solely in hardware. However, one of ordinary skill in the art, and based on a reading of this detailed description, would recognize that, in at least one embodiment, the electronic based aspects of the invention may be implemented in software. As such, it should be noted that a plurality of hardware and software based devices, as well as a plurality of different structural components may be utilized to implement the invention. Furthermore, and as described in subsequent paragraphs, the specific mechanical configurations illustrated in the drawings are intended to exemplify embodiments of the invention and that other alternative mechanical configurations are possible.

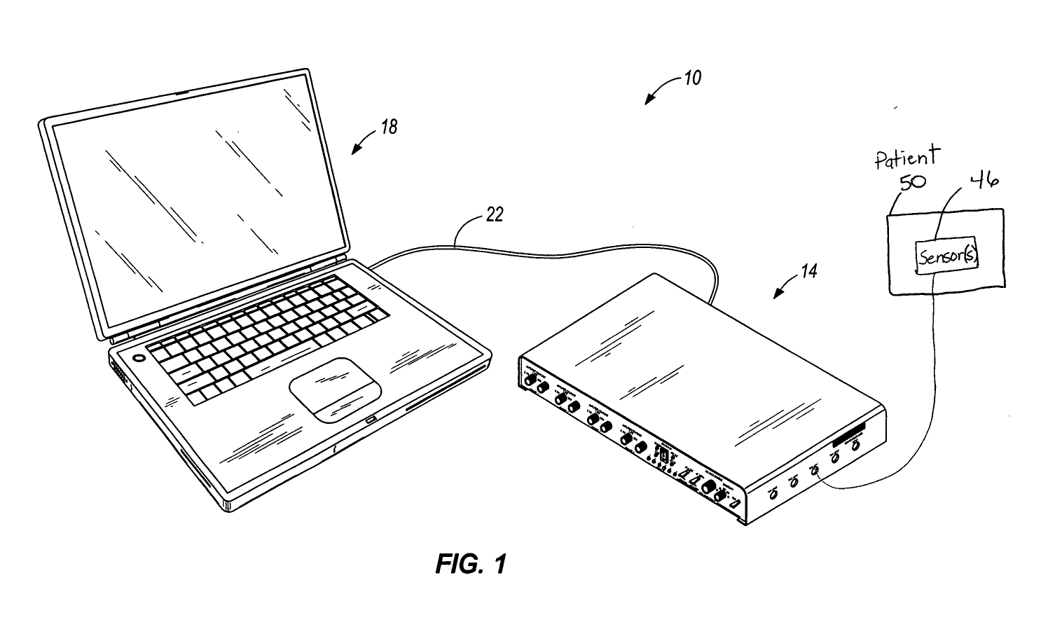

FIG. 1 illustrates a portable biofeedback system 10 according to one embodiment of the present invention. The portable biofeedback system 10 can be used for administering test procedures and experiments and acquiring psychophysiological data of a patient. The portable biofeedback system 10 includes a test module 14 and a personal computer 18, e.g., notebook computer or desktop computer. The test module 14 and the personal computer 18 are connected by a link 22, such as an electrical cord compatible with the output port of the test module 14 and the input port of the personal computer 18.

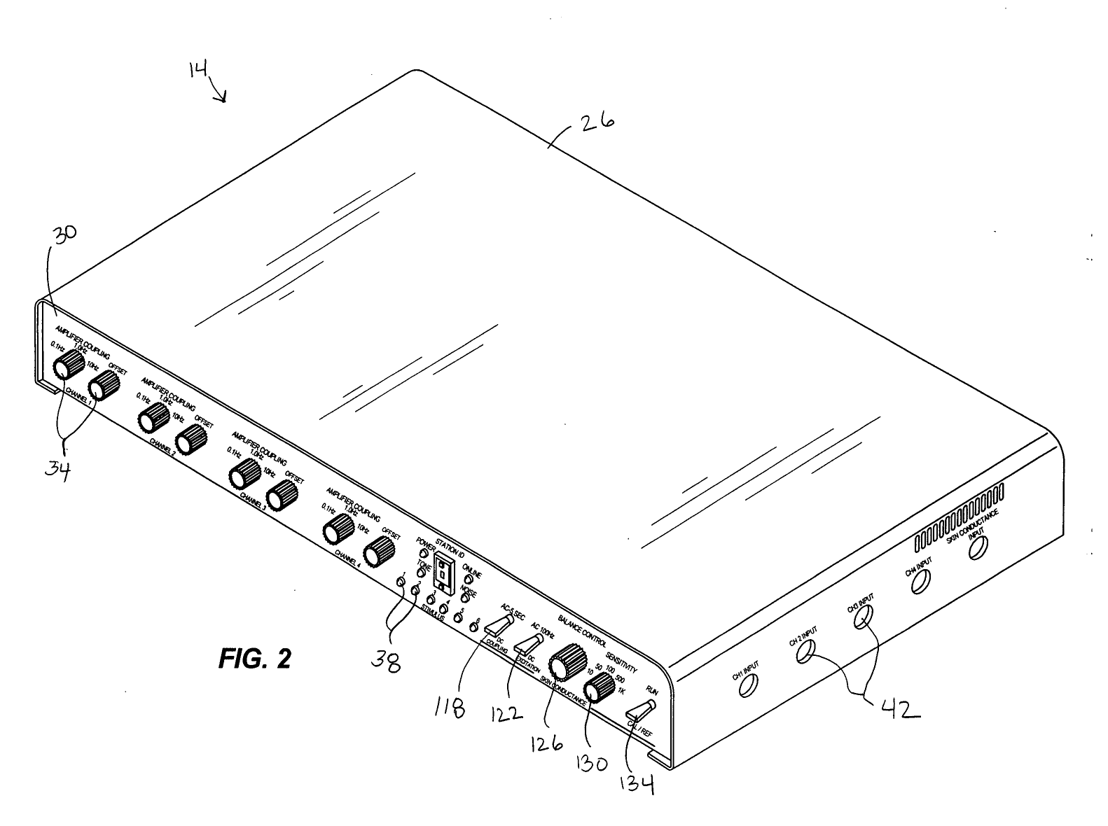

As illustrated in FIG. 2, the test module 14 includes a housing 26, defining an inner cavity, which supports electronic circuitry. The housing 26 includes a front panel 30, which supports a plurality of controls 34, a plurality of status indicators 38, and a plurality of input ports 42. The controls 34 can be any user manipulatable element or device, such as, a switch, a knob, a touch screen, a toggle, a slide, a button, and the like. FIG. 2 illustrates a particular number of controls 34, status indicators 38, and input ports 42, however, the test module 14 can be modified to include more or fewer controls 34, status indicators 38, and input ports 42 than illustrated. The controls 34 are user adjustable and operable to change or set signal processing parameters for acquired data. For example, the plurality of controls 34 can be used to adjust the amplifier coupling and offset for a plurality of channels of data. Each channel of data includes a control 34 for the amplifier coupling and a control 34 for the offset. The status indicators 38 can be used to indicate whether the test module 14 is receiving power, delivering a tone signal, is online, and/or delivering a noise signal. Additional status indicators 38 can be used to identify the particular stimuli used during the test. Additional controls 34 can be used to adjust the DC coupling, the DC excitation, balance, sensitivity, and introduction of a skin conductance calibration signal.

The input ports 42 are configured to receive a connector from a transducer or sensor 46, such as, an ECG electrode, an EEG electrode, a thermistor probe, a blood pressure transducer, and the like. The sensor(s) 46 is connected to a patient 50 (see FIG. 1), and the test module 14 is operable to acquire data from the patient 50 via the sensor(s) 46.

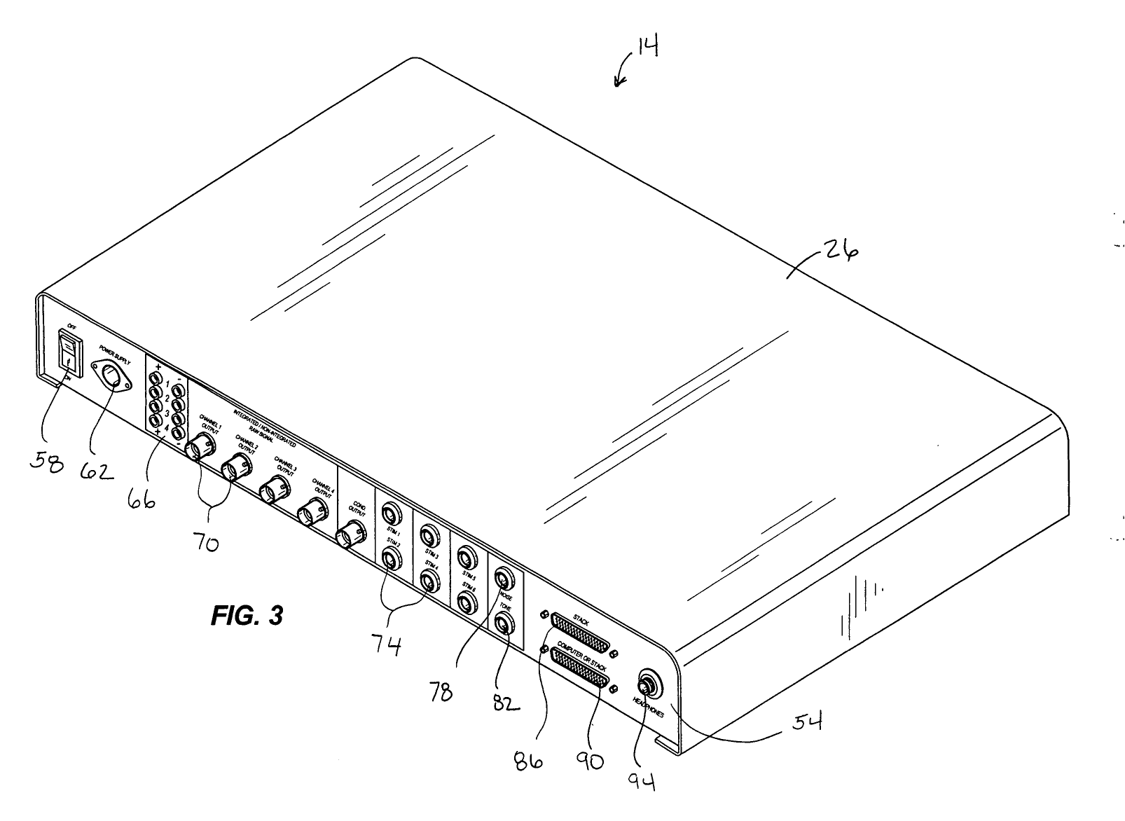

As illustrated in FIG. 3, the housing 26 includes a rear panel 54, which supports a plurality of input ports and output ports. The rear panel 54 includes an on/off switch 58 and a power supply input port 62 to receive an input connector from a power supply. The rear panel 54 also includes a plurality of remote inputs 66 that can provide a 5 to 30 VDC input signal to the electronic circuitry. The rear panel 54 also includes a plurality of channel outputs 70 that can provide a raw or integrated data signal to the personal computer 18 or other device. The rear panel 54 also includes a plurality of stimuli outputs 74 that can provide a signal to a stimulus delivery device, such as a shocker, a speaker, an air puff device, a lighting device, and/or an auxiliary relay that can connect to any type of stimulus delivery device. The stimuli outputs 74 can provide a 5 to 30 VDC output signal to the stimuli delivery devices. The rear panel 54 also includes a noise output 78 that can provide a noise signal, and a tone output 82 that can provide a tone signal. The rear panel 54 further includes an input 86 that can receive a connector from another test module 14. The rear panel 54 further includes an output 90 that can receive a connector from the personal computer 18. The rear panel also includes a headphone input 94 that can receive a connector from headphones.

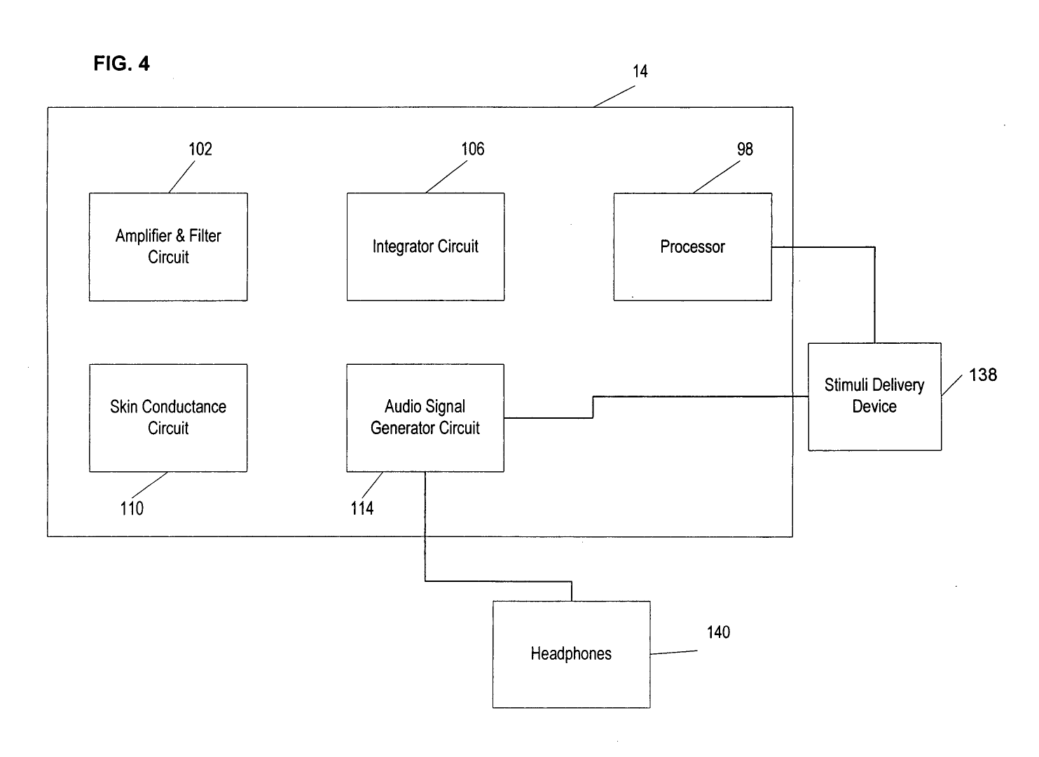

As shown schematically in FIG. 4, the test module 14 includes a processor 98 operable to acquire data from the patient 50. The test module 14 also includes an amplifier and filter circuit 102, an integrator circuit 106, a skin conductance circuit 110, and an audio signal generator circuit 114.

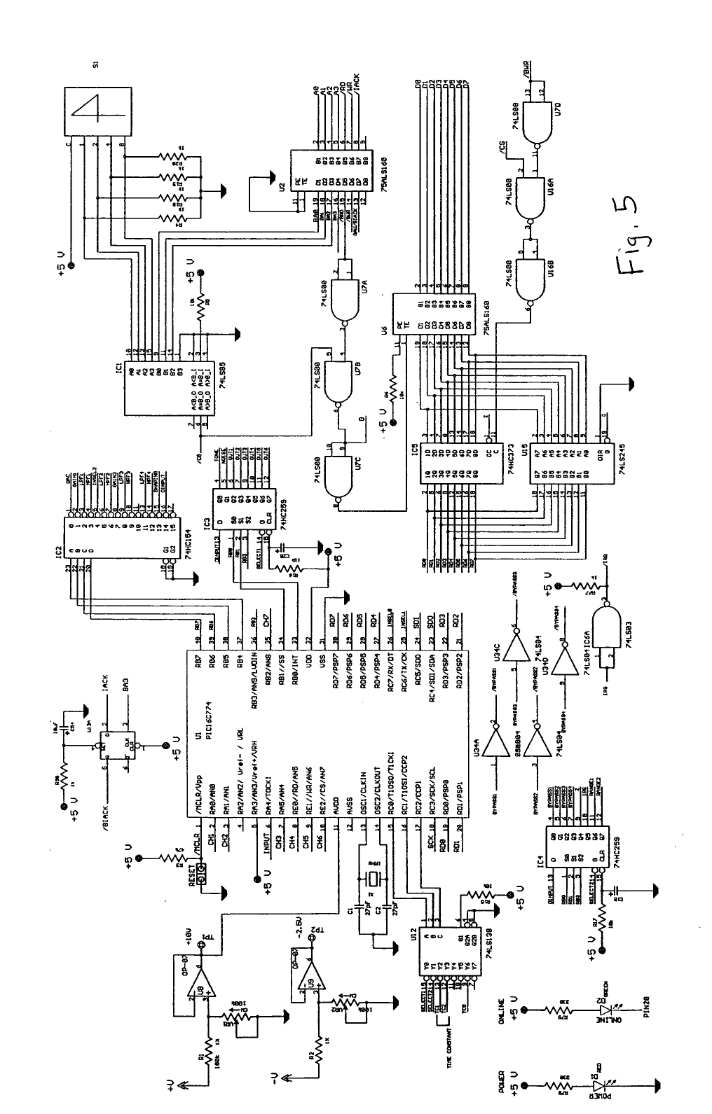

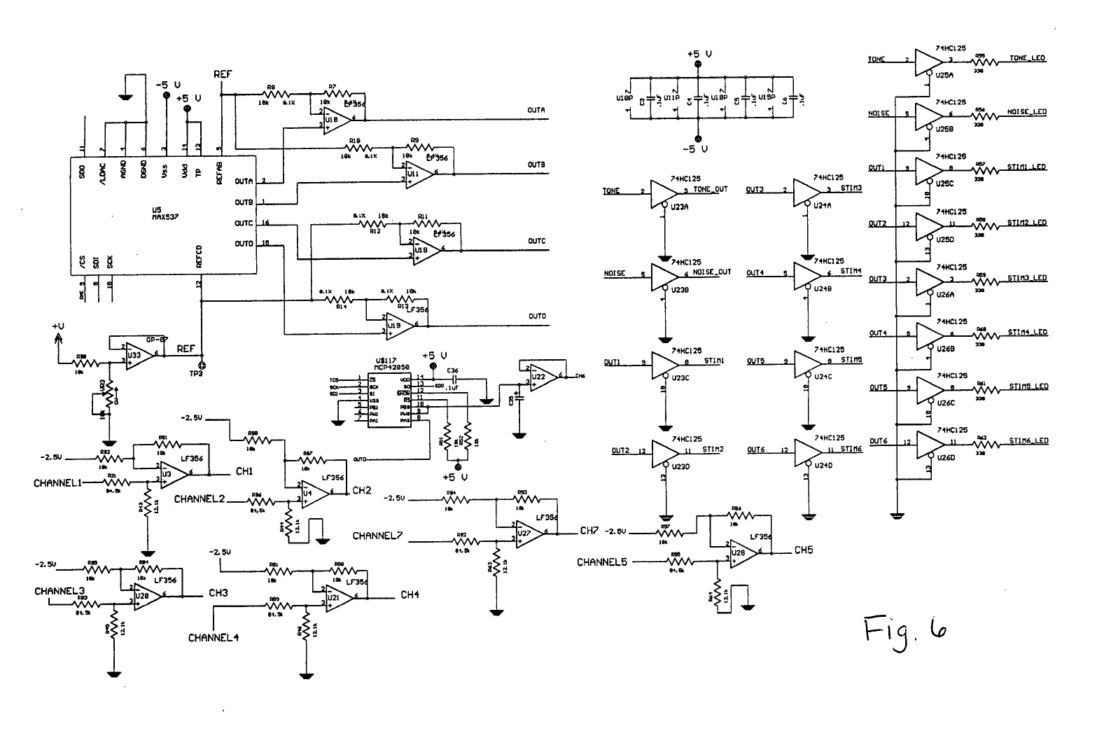

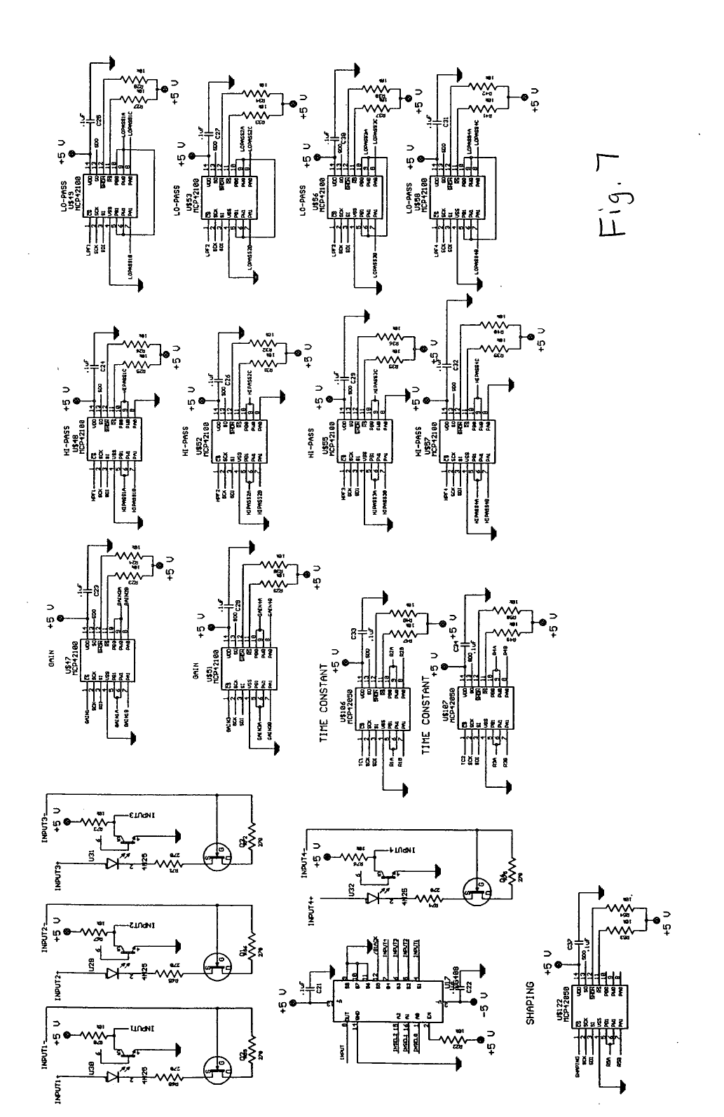

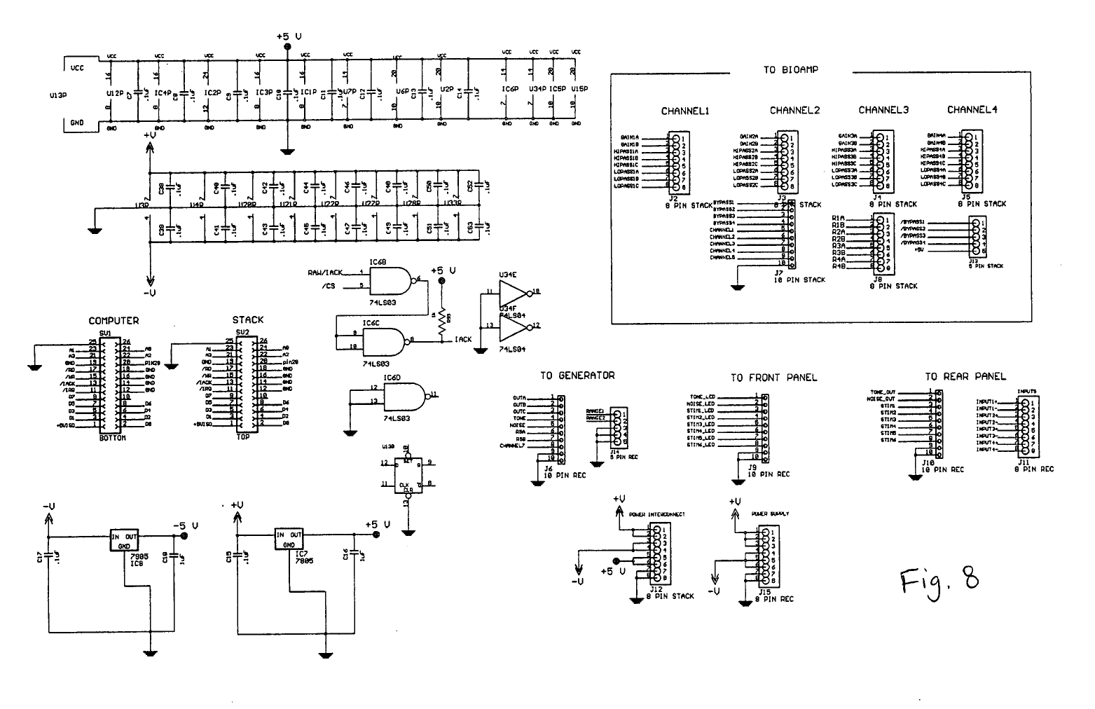

The processor 98, illustrated in more detail in FIGS. 5-8, is operable to transmit the acquired signals to the personal computer 18. The processor 98 communicates with the amplifier and filter circuit 102, the integrator circuit 106, the skin conductance circuit 110, the audio signal generator circuit 114, and/or the personal computer 18. The processor 98 is operable to drive a plurality of stimuli delivery devices 138 to provide various stimuli to the patient 50. The processor 98 can control the actuation of the stimuli delivery devices 138 by providing a 5 to 30 VDC output signal to the stimuli outputs 74.

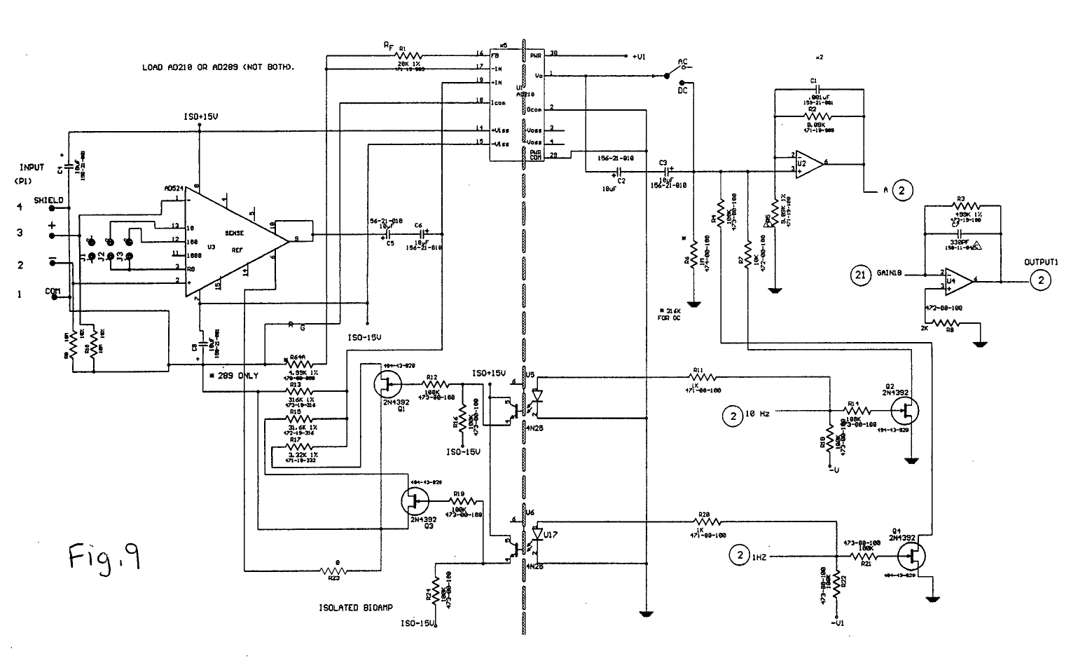

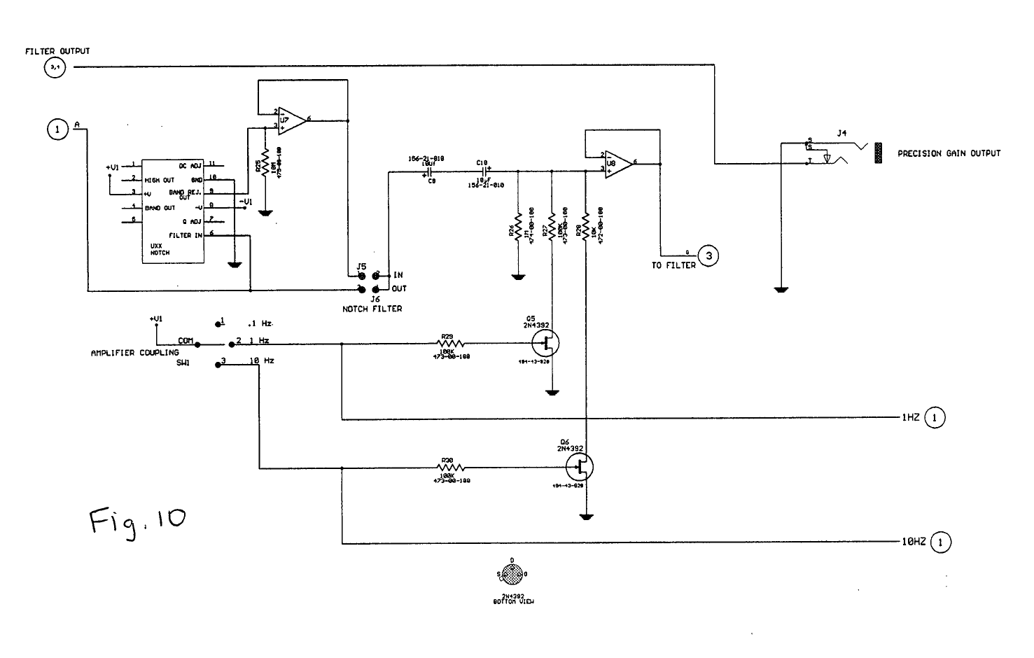

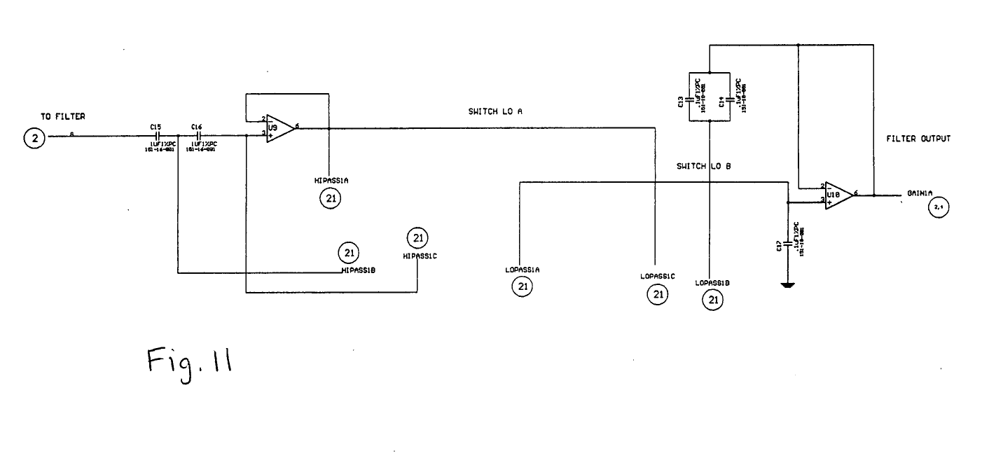

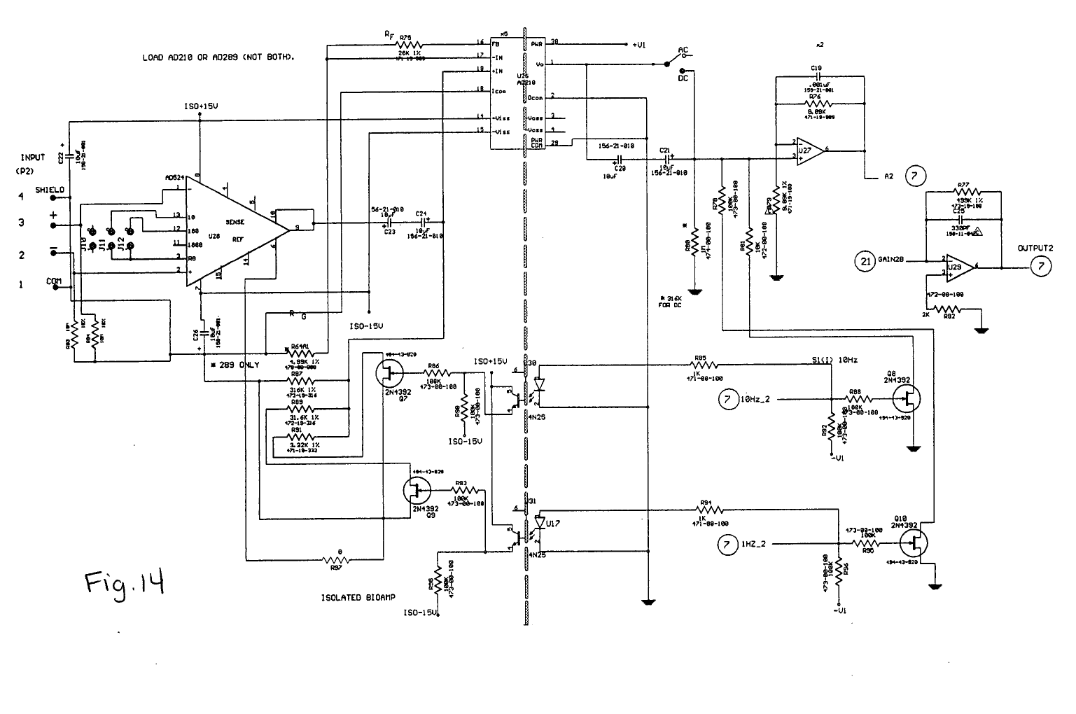

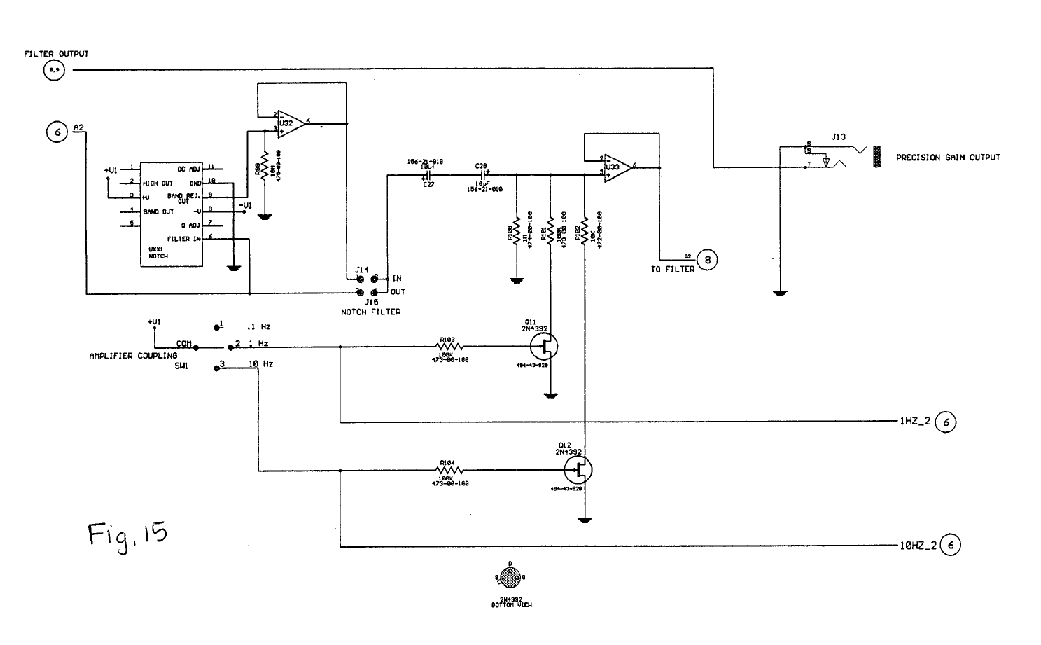

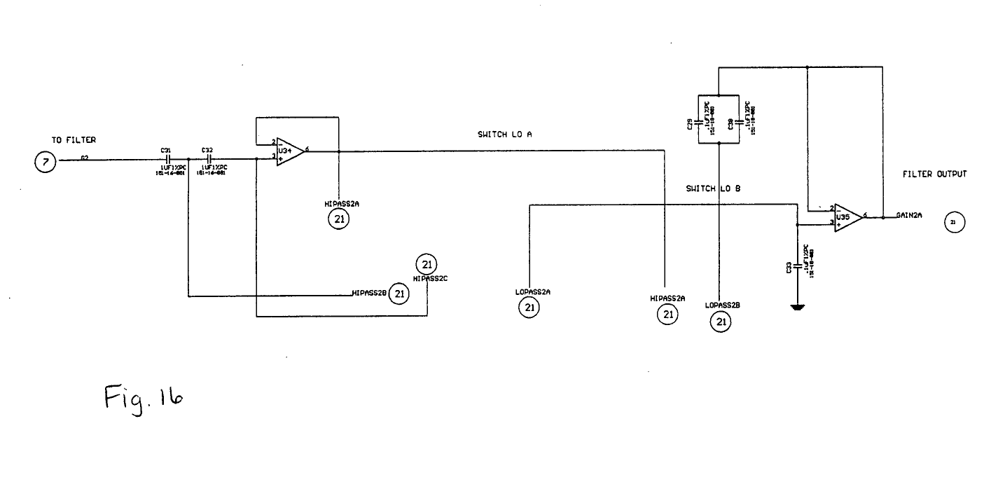

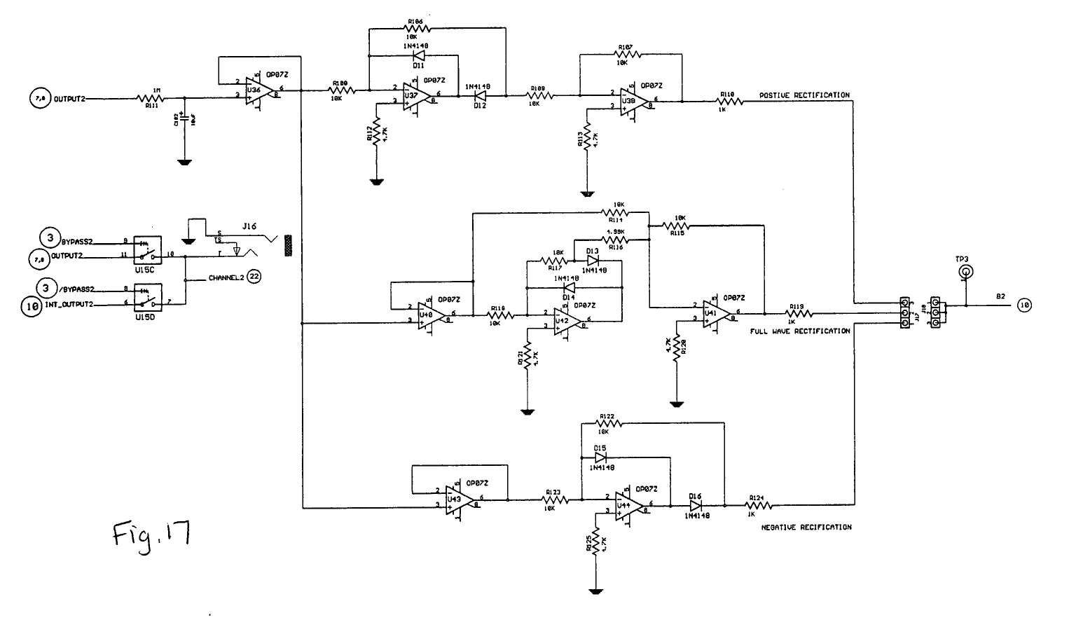

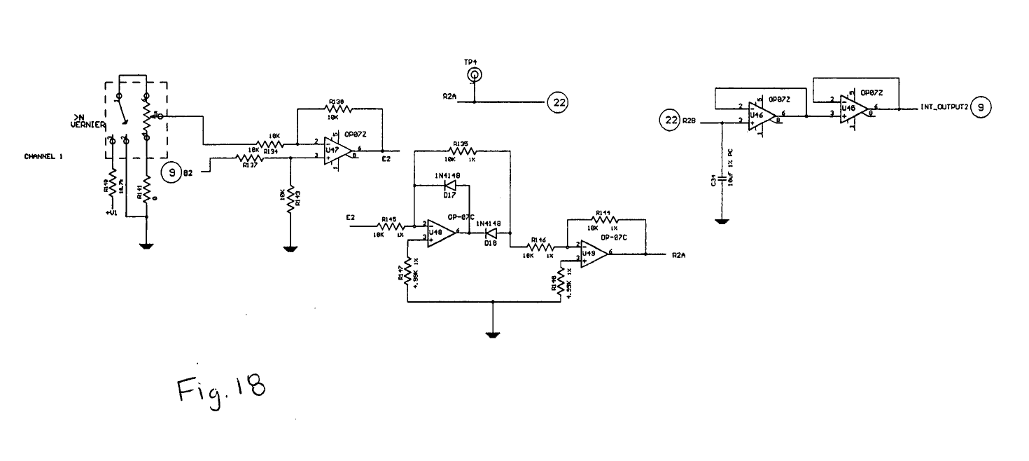

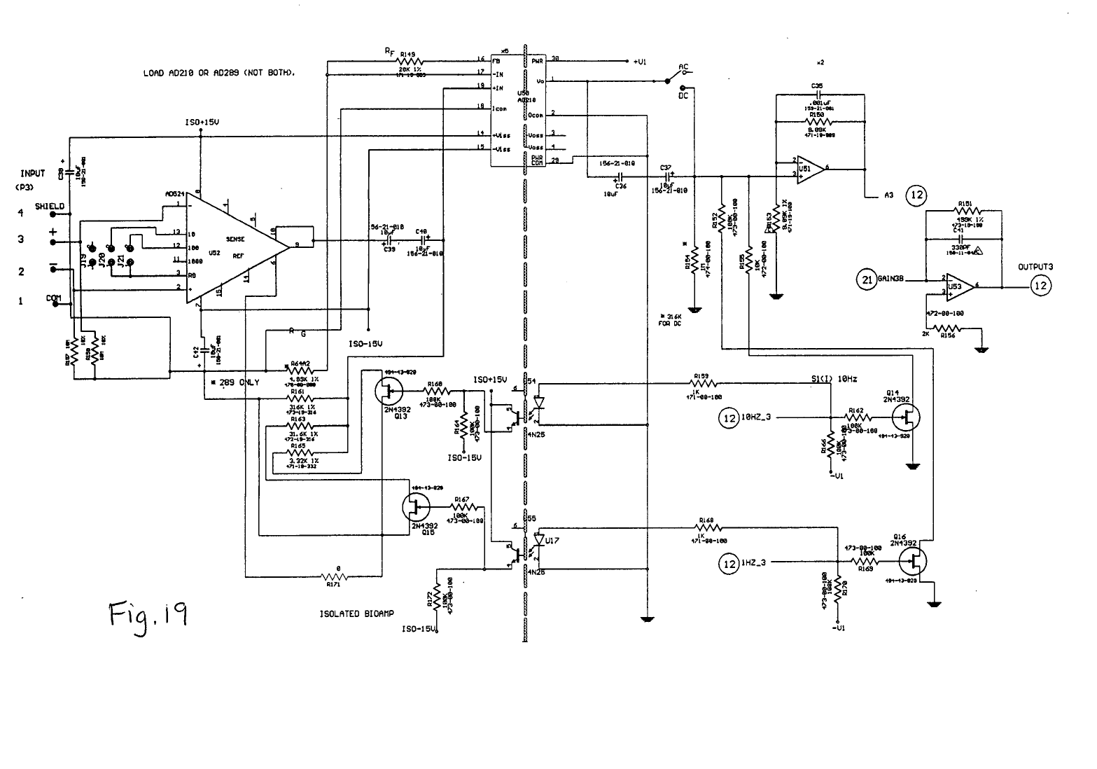

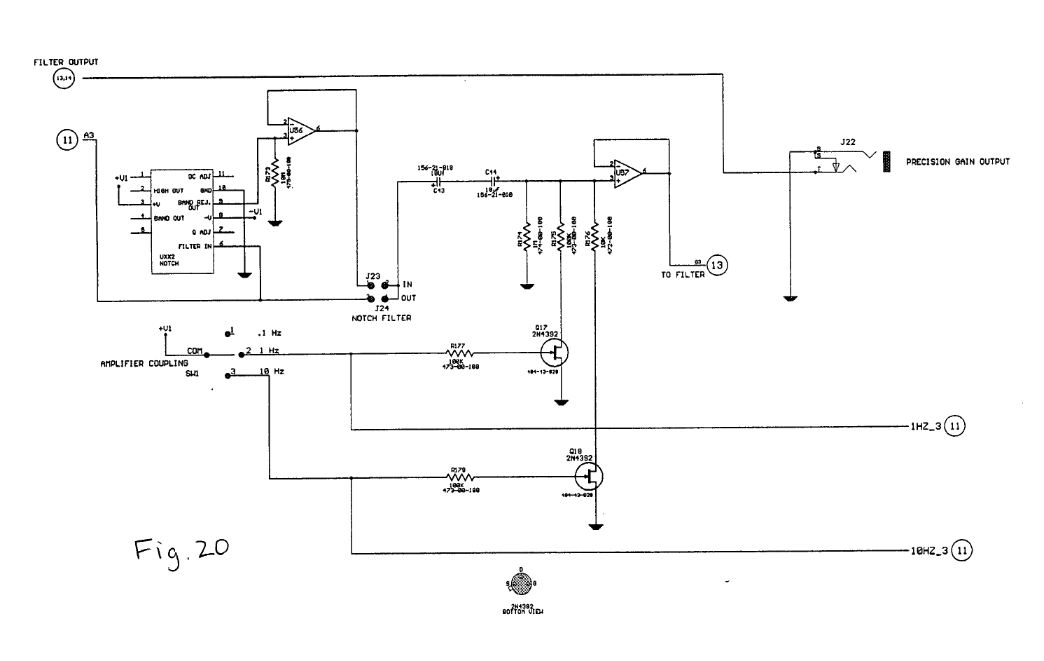

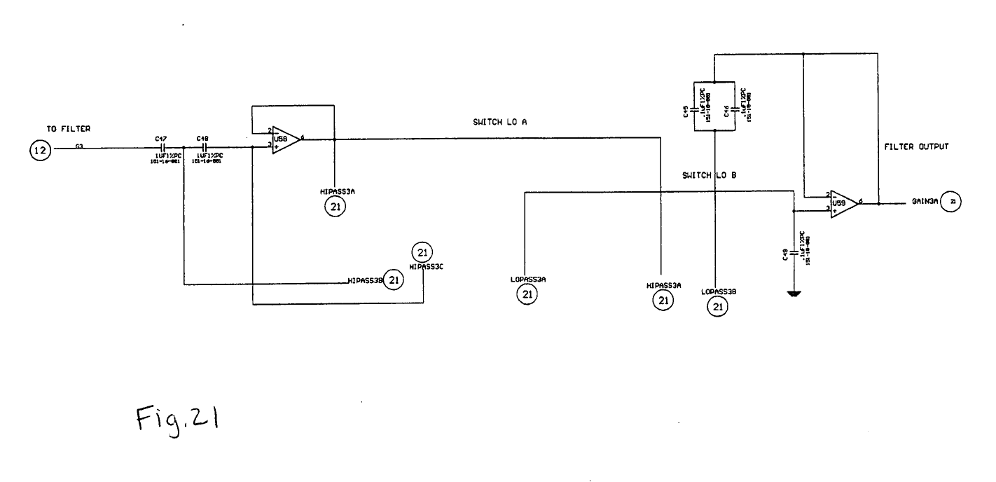

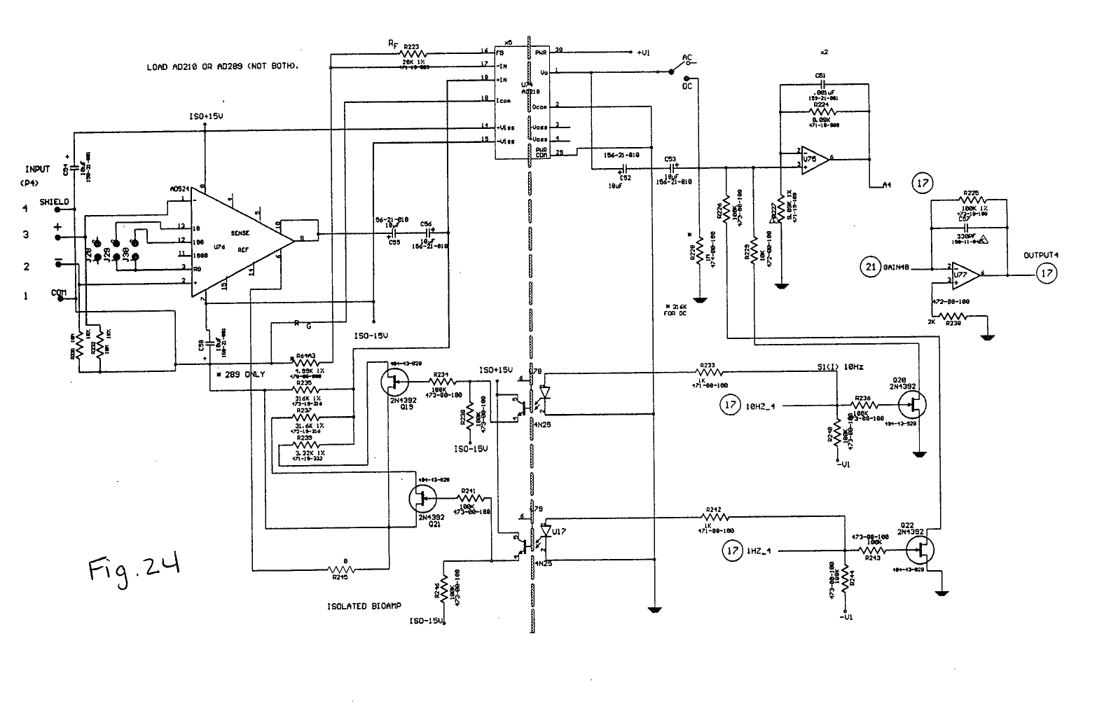

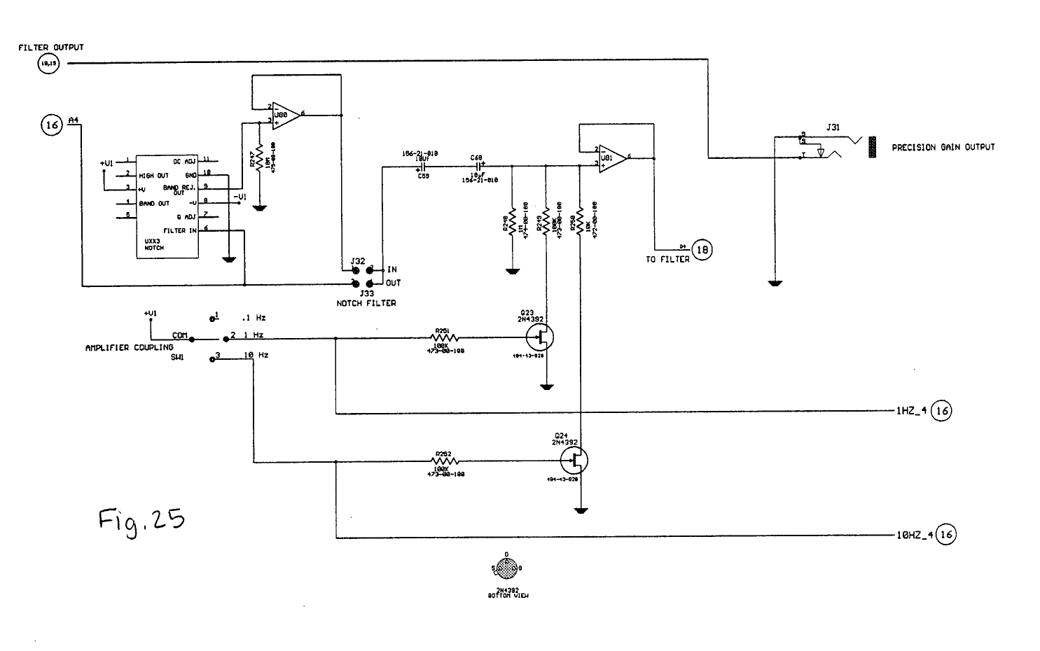

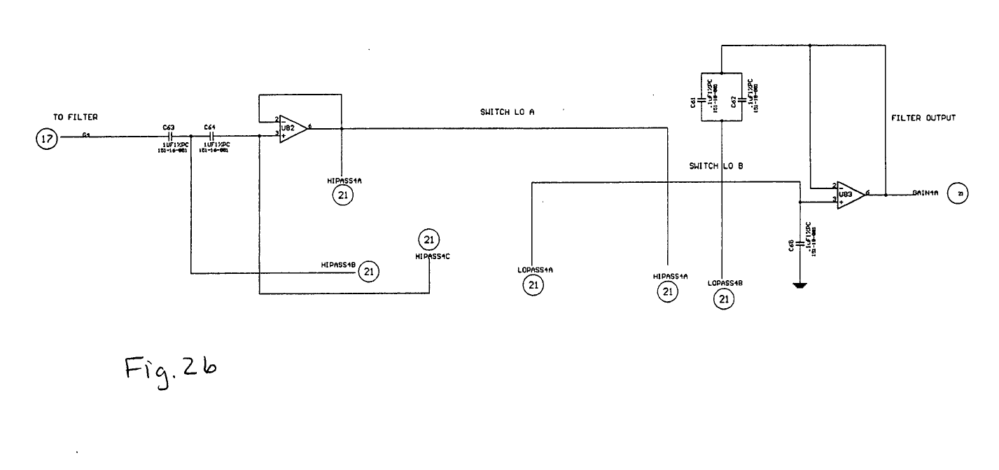

The amplifier and filter circuit 102, illustrated in more detail in FIGS. 9-11, 14-16, 19-21, and 24-26, is operable to amplify and filter the data signals, e.g., ECG, EMG, EEG, etc., acquired from the patient 50. The amplifier and filter circuit 102 comprises a bandpass filter network including a plurality of low-pass and high-pass filter frequency settings (e.g., 5 low-pass and 5 high pass). The amplifier and filter circuit 102 also includes a 60 (or 50) Hz notch filter and a coupler filter between a preamplifier and a main amplifier section to permit decoupling when artifact-caused saturation is present. The preamplifier coupling circuit also extends the range of the low cutoff side of the pass band. Setting the hi-pass (low cutoff) filter to the lowest setting (open to the preamplifier) allows the coupling filter to set the low cut skirt of the bandpass filter. The filters are 2-pole Butterworth type and provide 12 dB per octave attenuation for EMG, ECG, EEG, SPR, and other acquired data signals. In some embodiments of the invention, the amplifier and filter circuit 102 can be separate circuits. The filters can be manually adjusted with the appropriate control 34 and/or via a software program (discussed below).

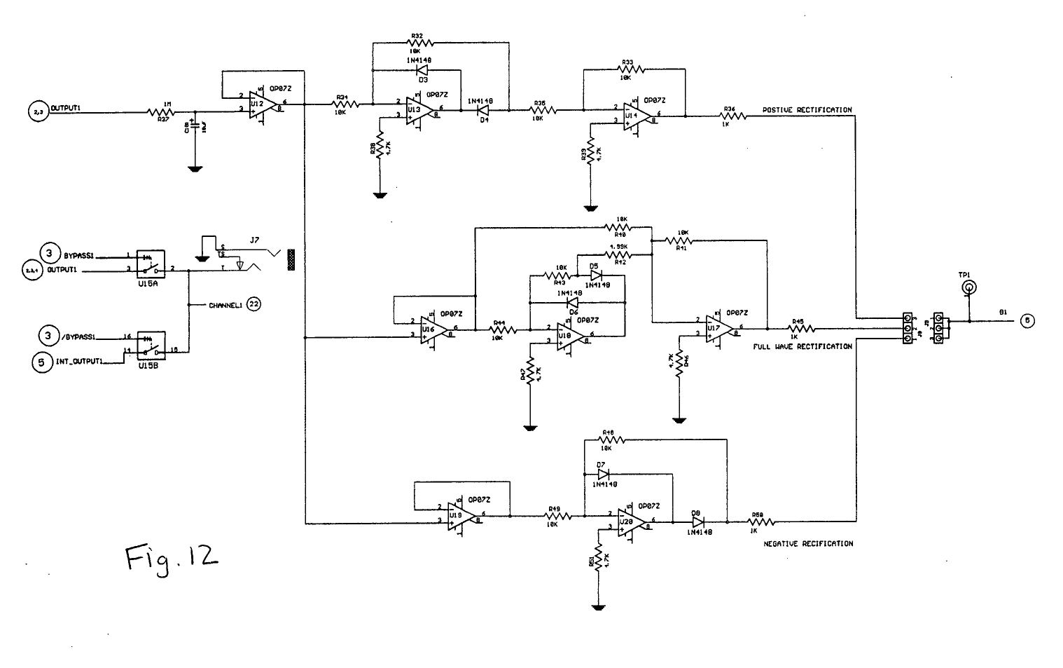

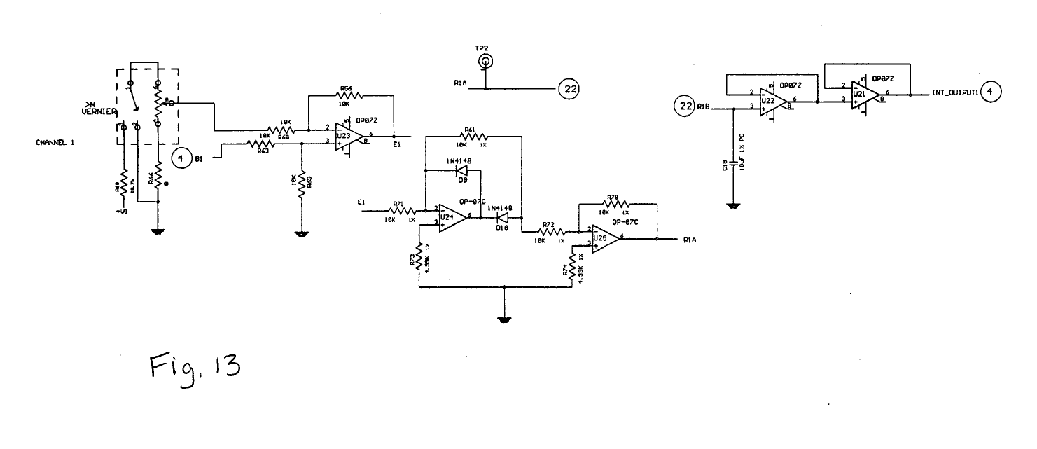

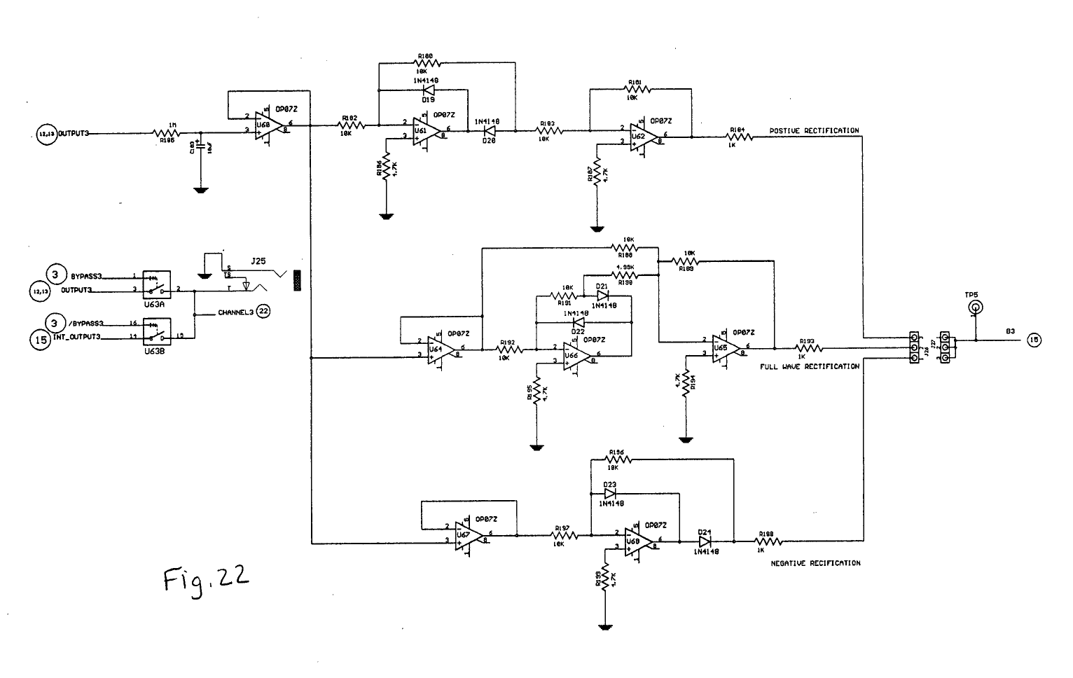

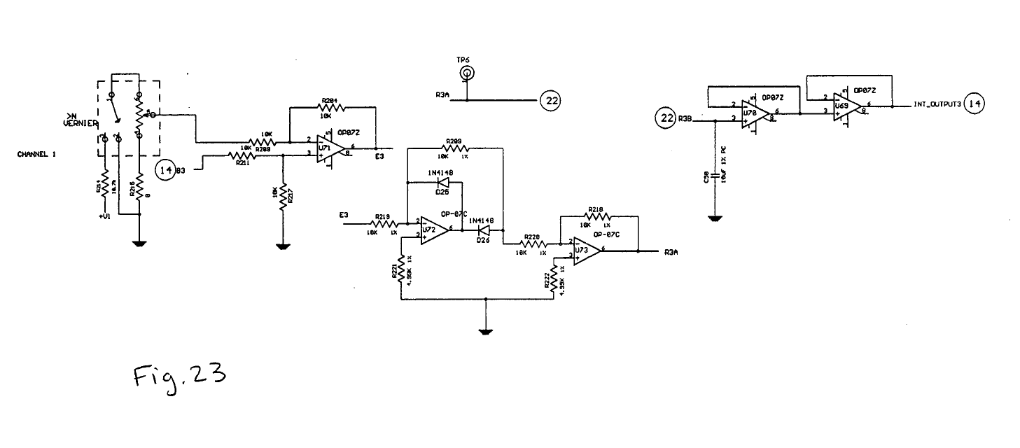

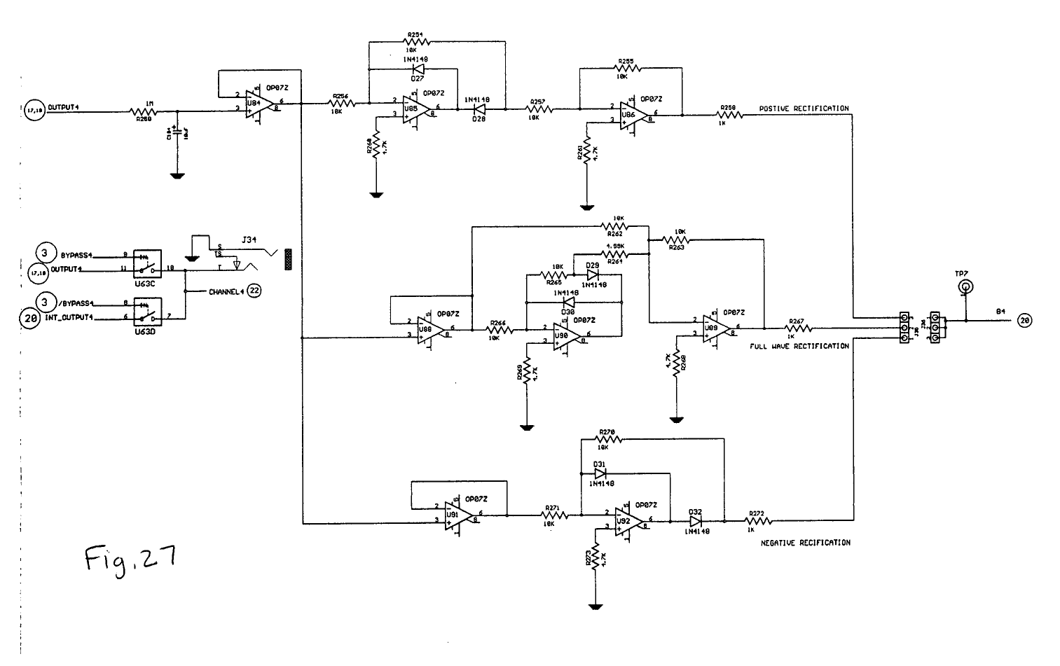

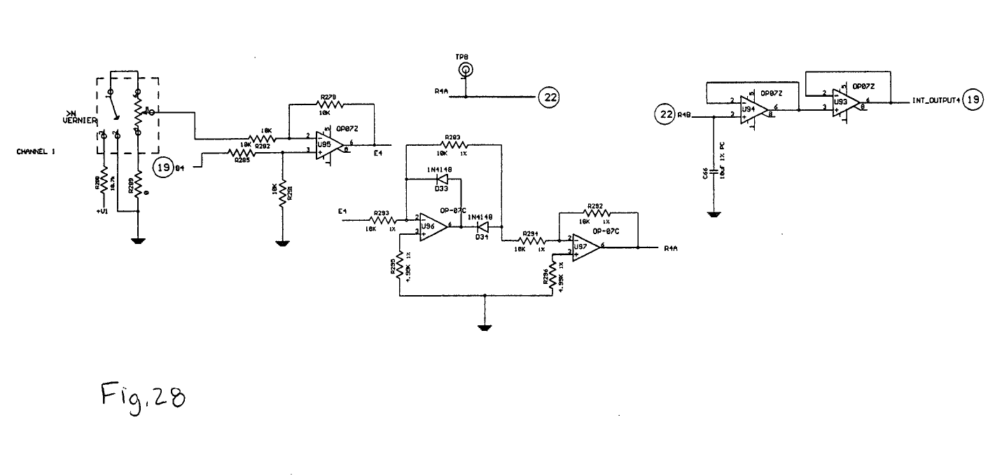

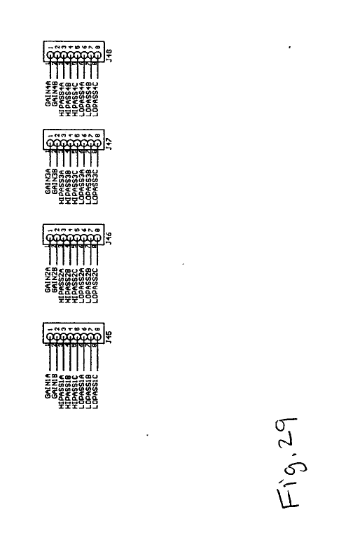

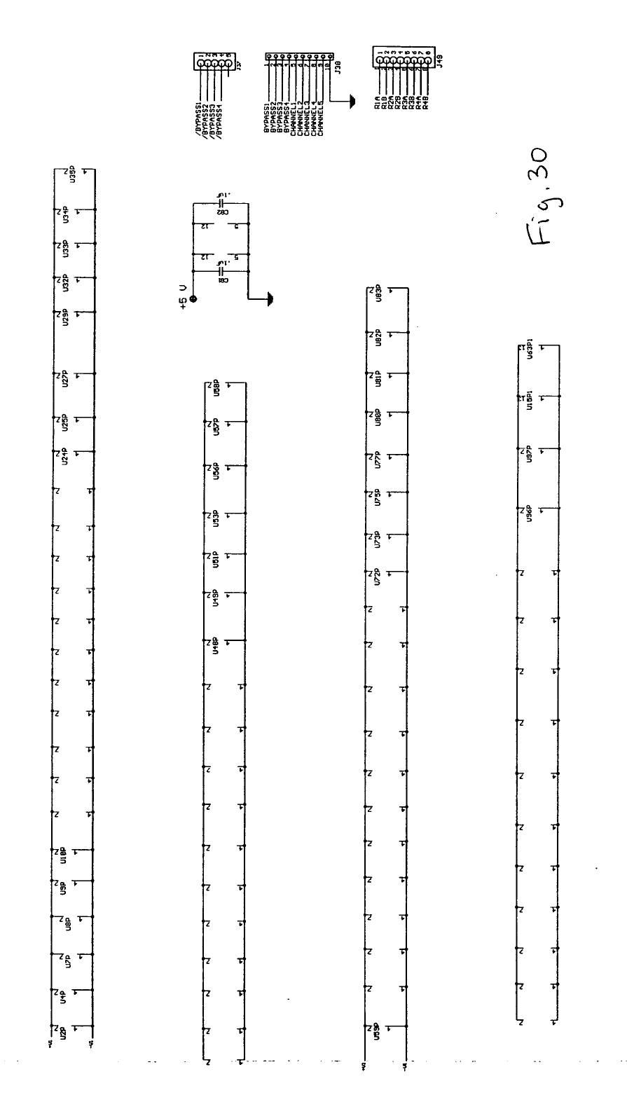

The integrator circuit 106, illustrated in more detail in FIGS. 12-13, 17-18, 22-23, and 27-28, comprises a plurality of independently controlled contour following integration channels. A contour following integrator generally is a rectifier and a single-pole low pass filter. The rectifier section rectifies “full-wave” or “half-wave” for either the positive or negative component of bipolar signals. The integrator circuit 106 is operable to provide multi-channel integration display. Each channel has a time constant rotary switch, negative offset adjust and on board jumpers for positive, negative and full-wave rectification. The contour following integrator is used to “smooth out” high frequency signals such as EMG signals where the contour or “envelope” of the signal is of interest to the user. The integrator circuit 106 also includes a cumulating/resetting circuit. The cumulating/resetting circuit and the contour following integrator operate simultaneously and independently. A time constant can be set independently and each circuit has its own output. The cumulating/resetting circuit is operable to provide a precise measure of the area under the curve as the root mean square of the instantaneous amplitude of the signal. The integrator circuit 106 is also operable to record aspects of such signals without impossibly high analog-to-digital conversion rates, large data files, and massive amounts of statistical processing. The integrator circuit 106 is also operable to provide the viewing of meaningful data in real time with computer-based software. The negative offset allows the user to “zero out” background noise and/or unwanted baseline signal coming from the input source. The negative offset can also be used to offset subject baseline response or “tonic level” as well as to eliminate other noise and background signal that should not appear in the record.

The test module 14 can include a plurality of amplifier and filter circuits 102 and integrator circuits 106. Generally, the test module 14 includes an amplifier and filter circuit 102 and integrator circuit 106 for each channel of data to be acquired. For example, the embodiment of the test module 14 illustrated in FIG. 2 includes four separate channels via which data can be acquired and output to the personal computer 18. Thus, the embodiment would include a separate amplifier and filter circuit 102 and a separate integrator circuit 106 for each channel.

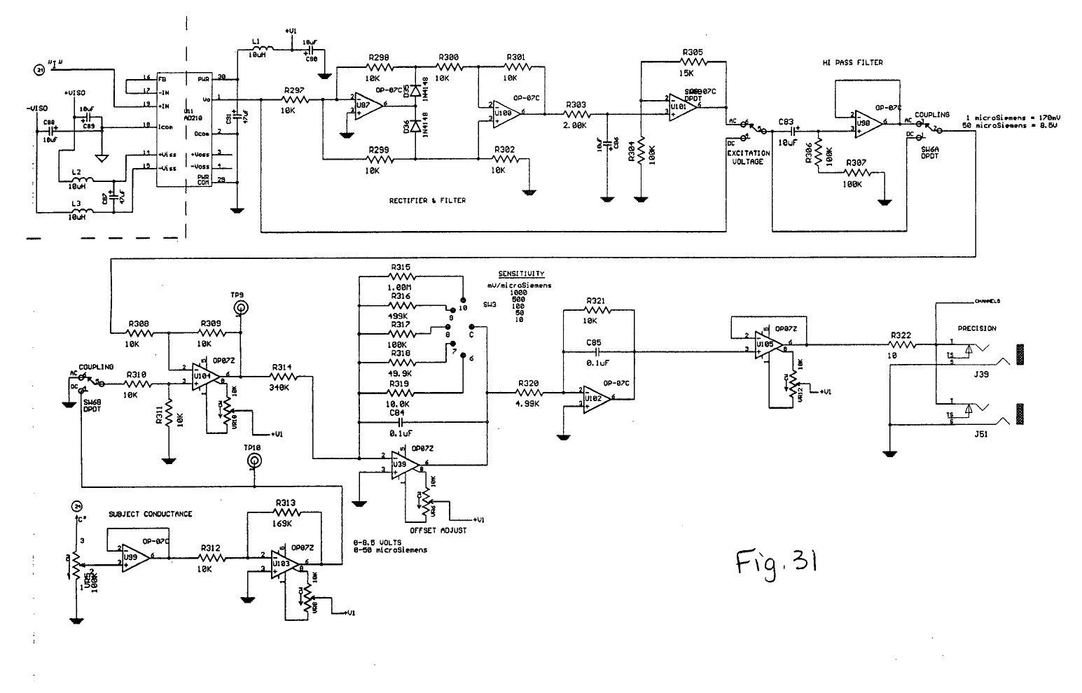

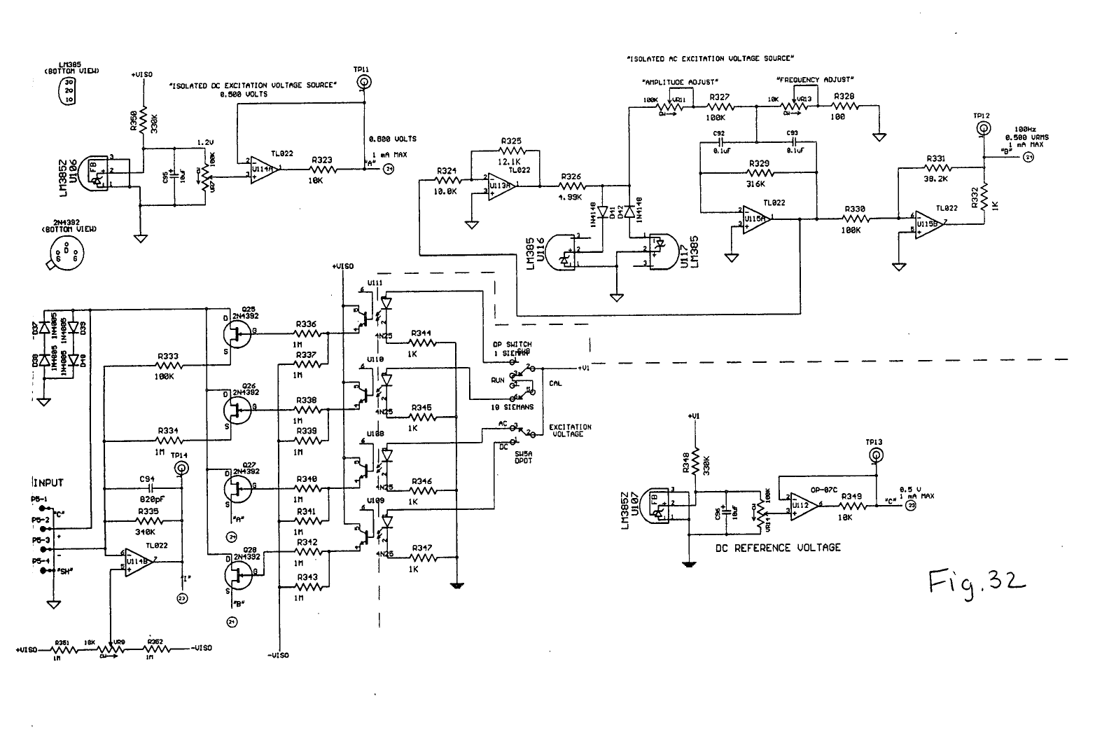

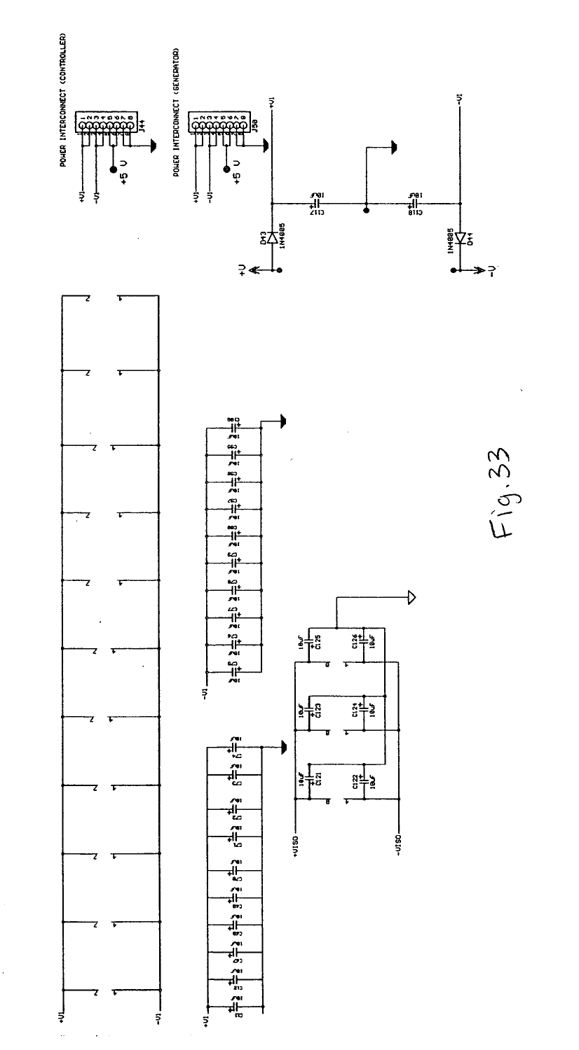

The skin conductance circuit 110, illustrated in more detail in FIGS. 31-33, is operable to measure skin conductance (the reciprocal of skin resistance). The skin conductance measurement also provides a measurement of the electrodermal response (“EDR”). It is noted that EDR is the collective/generic name for galvanic skin response, skin potential response, skin resistance, and skin conductance.

The skin conductance circuit 110 includes a coupling control 118 (see FIG. 2) that allows the user to select either direct coupling (DC) for the measurement of basaltonic—skin conductance level (“SCL”), or AC coupling to measure the skin conductance response (“SCR”). The SCR time constant can be five seconds. The skin conductance circuit 110 also includes a control 122 that allows the user to select the signal to be applied to the patient's skin. The control 122 allows the user to select between a 0.5 volt AC, low distortion sine signal or a 0.5 volt DC excitation signal. The resulting current flow (typically in the 5 microamp range) is processed by the coupler into an output voltage signal. The skin conductance circuit 110 also includes a balance control 126 that is user manipulatable. The balance control 126 is operable to balance the tonic (basal) conductance level of the patient 50 to zero when the unit is in the DC coupled mode. The skin conductance circuit 110 also includes a sensitivity control 130 that is user manipulatable. The sensitivity control 130 includes a calibrated output with a plurality of ranges. The skin conductance circuit 110 also includes a calibrate reference control 134 that is user manipulatable. The user can select the calibration signal (e.g., 1 μSiemens or 10 μSiemens) that can be introduced to the coupler input before, or during the time that the patient 50 is connected.

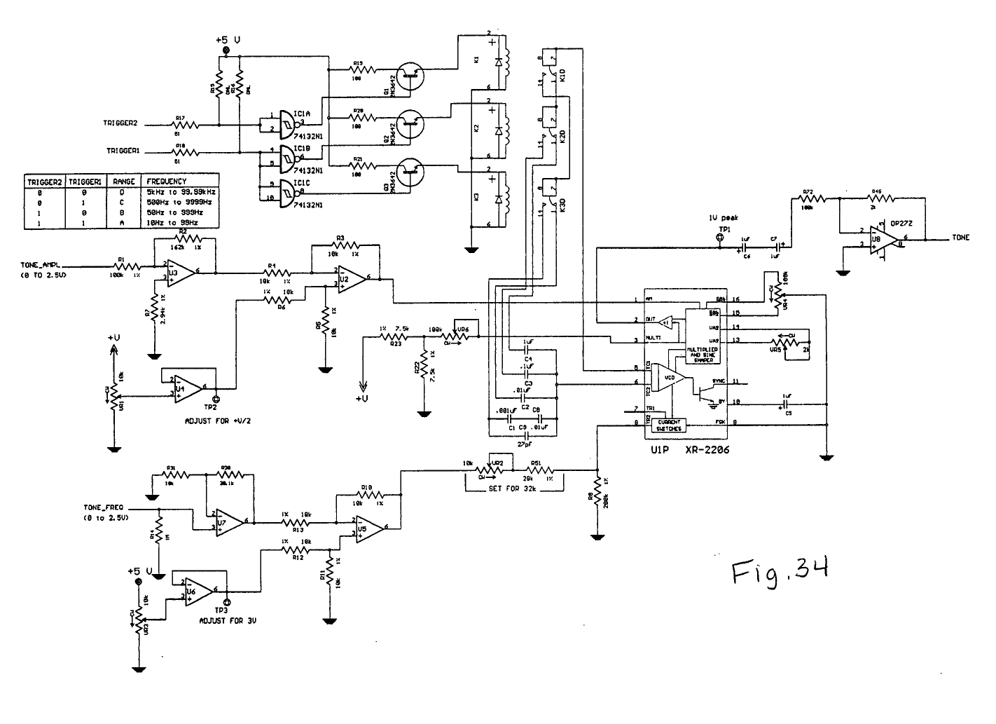

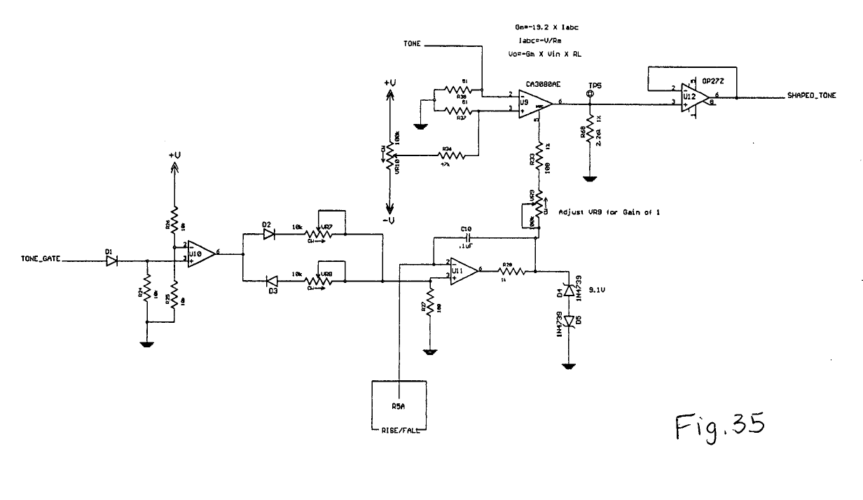

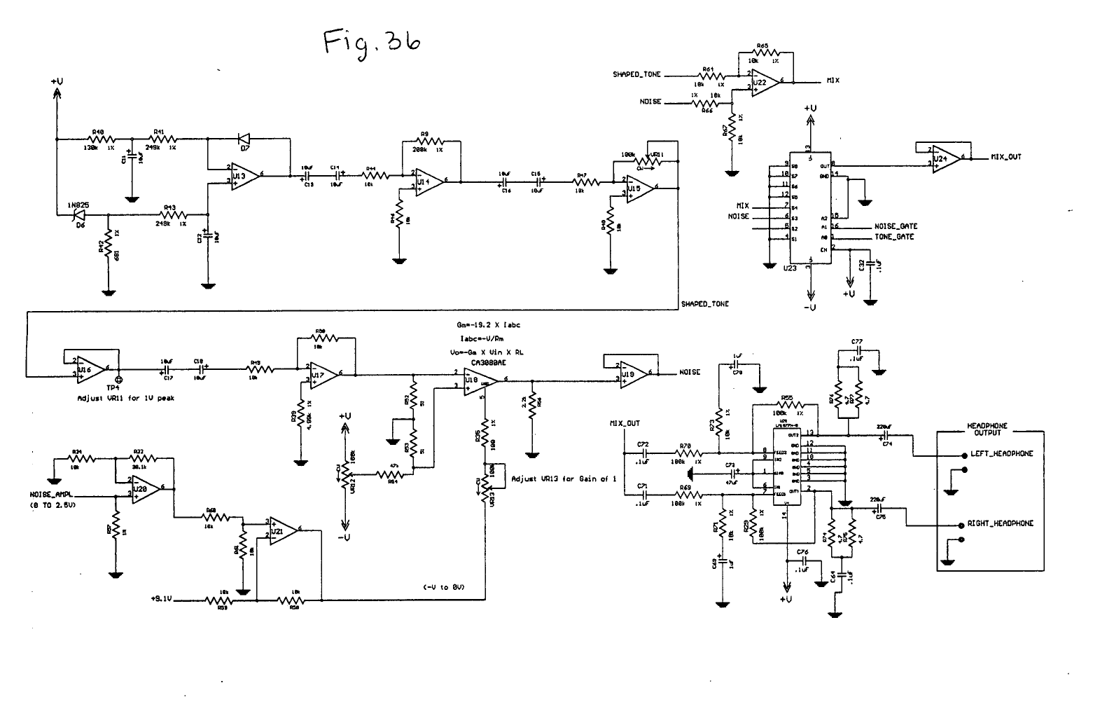

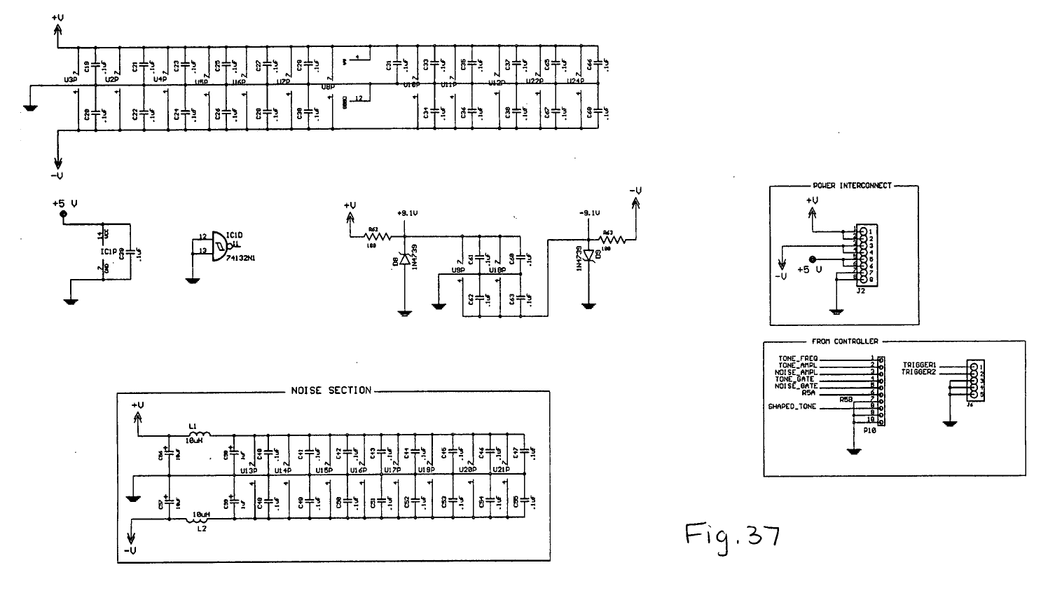

The audio signal generator circuit 114, illustrated in more detail in FIGS. 34-37, is operable to drive a plurality of stimuli delivery devices 138 to provide various stimuli to the patient 50. The audio signal generator circuit 114 includes a signal generator with a headphone driver operable to drive headphones 140 to provide a noise signal and/or a tone signal to the patient 50. The headphones 140 can deliver various tone signals and noise signals to the patient 50 to measure a startle reflex of the patient 50. The patient's startle reflex is reflected in the data acquired from the patient 50, e.g., EEG, ECG, and EMG. The audio signal generator circuit 114 includes a tone frequency circuit 142, a tone amplitude circuit 146, a noise amplitude circuit 150, and a duration circuit 154 that are all user manipulatable via a software program (discussed below).

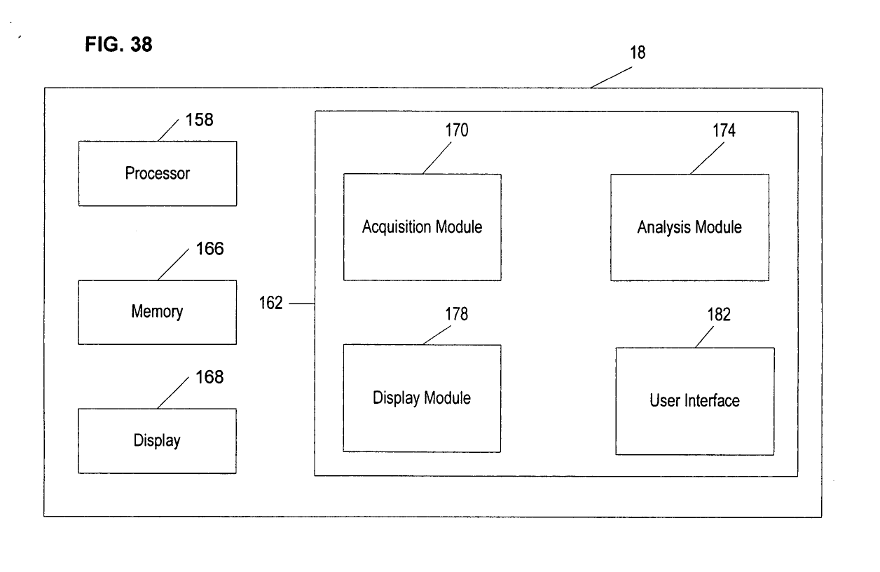

FIG. 38 schematically illustrates one embodiment of the personal computer 18 of the present invention. The personal computer 18 includes a processor 158 operable to run a software program 162. The personal computer 18 also includes a storage device or memory 166. The personal computer 18 can include additional hardware, such as 10 interfaces and additional storage devices or memory. The personal computer 18 can also include input devices (such as a keyboard and a mouse) and output devices (such as a monitor or display 168). The personal computer 18 can further include peripherals (such as a printer and a scanner). The personal computer 18 can still further include other software, such as an operating system, communications application, and a display application.

The software program 162 includes an acquisition module 170, an analysis module 174, a display module 178, and a user interface 182. The acquisition module 170 is operable to receive the data from a plurality of test modules 14. The acquisition module 170 also is operable to receive data from other modules, devices, and systems. The analysis module 174 is operable to receive the data from the acquisition module 170 and can analyze the data. The analysis module 174 can output the analyzed data to the display 168 of the personal computer 18. The display module 178 is operable to configure or manipulate the data for display on the monitor 168 of the personal computer 18.

The user interface 182 includes a plurality of screens or pages, viewable on the personal computer 18, that can be browsed by the user. The terms “screen” and “page” can refer to any grouping or association of data regardless of the presentation formatting or programming used to create the grouping or association. As such, all of the screens of the user interface 182 are not limited to the arrangement as shown in any of the drawings. The screens may include, but are not limited to fields, dialog boxes, tabs, buttons, radio buttons, and drop down menus. Field titles may vary and are not limited to that shown in the drawings.

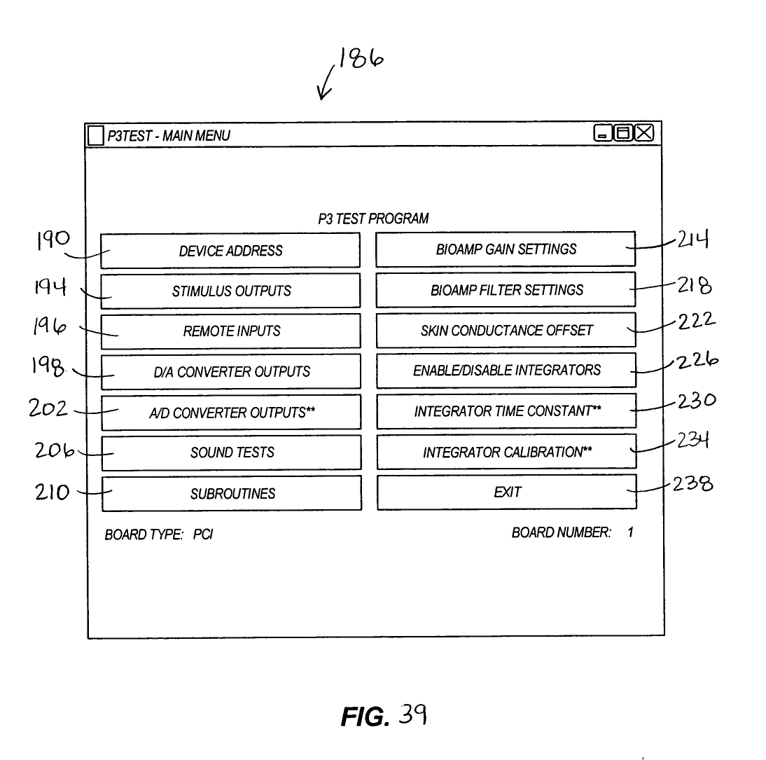

FIG. 39 illustrates a main screen 186. The main screen 186 includes a plurality of options for the user. The user can select any one of the options and a new screen will appear. The main screen 186 includes a device address selection 190, a stimulus outputs selection 194, a remote inputs selection 196, a D/A converter outputs selection 198, an A/D converter outputs selection 202, a sound tests selection 206, a subroutines selection 210, a bioamp gain settings selection 214, a bioamp filter settings selection 218, a skin conductance offset selection 222, an enable/disable integrators selection 226, an integrator time constant selection 230, an integrator calibration selection 234, and an exit selection 238.

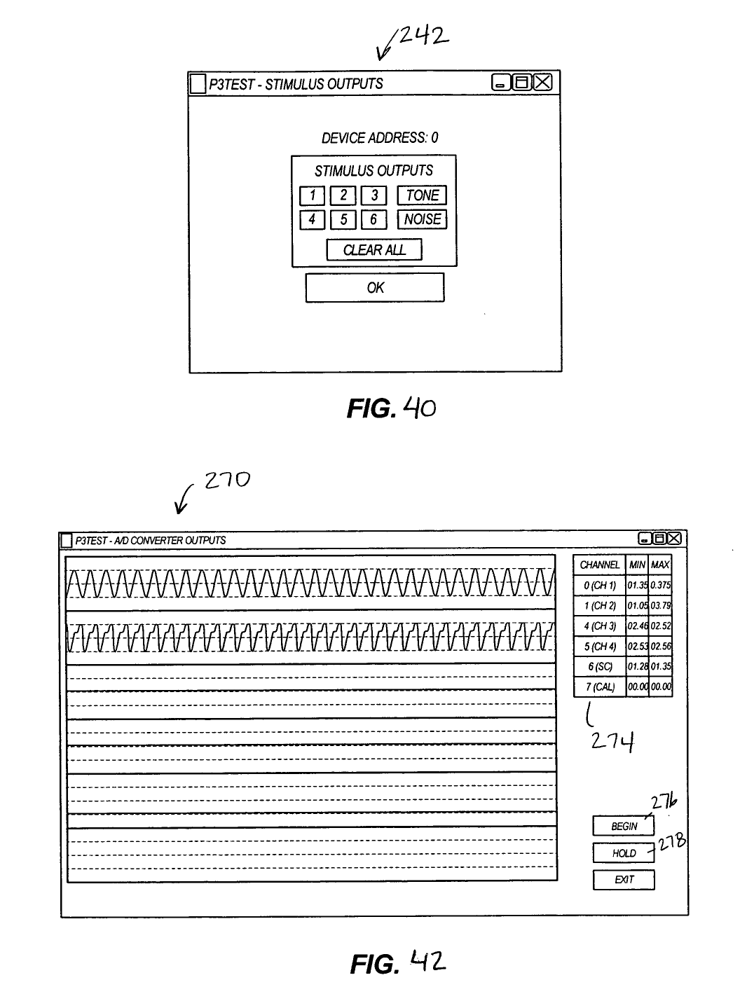

The stimulus outputs selection 194 links to a stimulus outputs screen 242 illustrated in FIG. 40. The user can select between a plurality of stimulus outputs, a tone output, and a noise output. The user can connect the selected stimuli delivery devices 138 to the stimuli outputs 74 on the test module 14. The user can access the stimulus outputs screen 242 to configure any of the stimulus output parameters of the stimuli delivery devices 138. Any test or trial can include a pure tone and/or white noise stimulus and either can appear in the pre-pulse or startle stimulus temporal position. Blank trials (e.g., trials during which neither stimulus is presented) can also be included. For tones, the user can specify the duration, the amplitude and the frequency (discussed below). For white noise, the user can specify the duration and amplitude (discussed below).

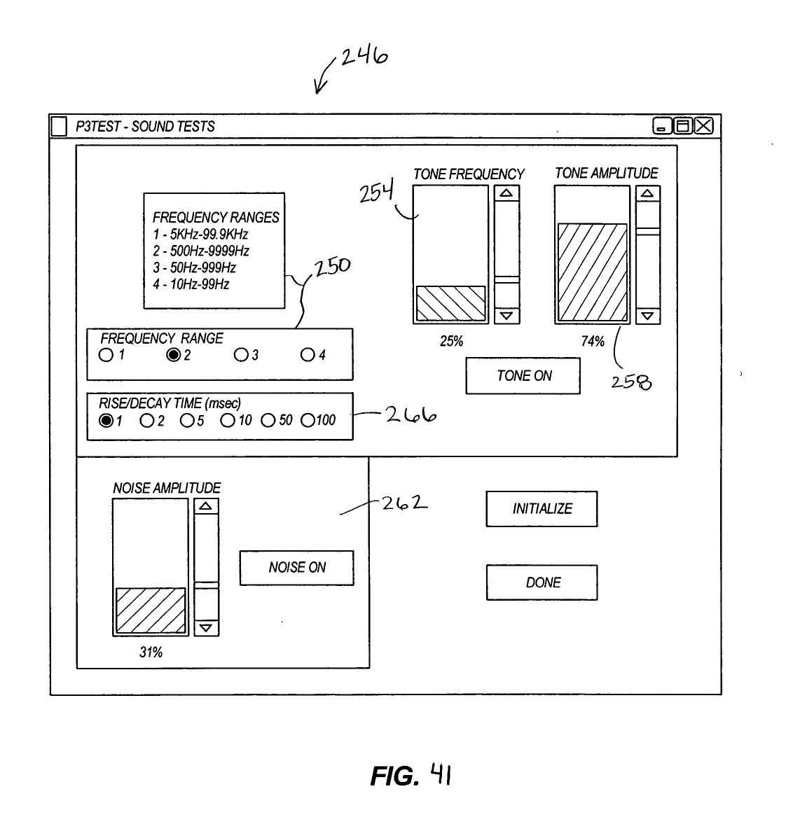

The sound tests selection 206 links to a sound tests screen 246 illustrated in FIG. 41. The sound tests screen 246 includes a frequency range section 250 including particular frequency ranges that the user can select for the tone of the startle reflex test(s) to be performed on the patient 50. The embodiment of the sound tests screen 246 illustrated in FIG. 41 identifies specific frequency values and ranges of frequency values that the user can select. It is noted that the specific frequency values and ranges of frequency values can change and the invention is not limited to the frequency values and the ranges of frequency values illustrated in FIG. 41. The sound tests screen 246 also includes a tone frequency section 254, a tone amplitude section 258, a noise amplitude section 262, and a signal duration section 266. As noted above, the user can adjust the parameters of the tone or noise to be generated by the audio signal generator circuit 114 and delivered to the patient 50 via headphones or speaker. The user can select and/or adjust the frequency, amplitude, and duration of the tone signal in the tone frequency section 254, the tone amplitude section 258, and the signal duration section 266, respectively. The user can select and/or adjust the amplitude and the duration of the noise signal in the noise amplitude section 262 and the signal duration section 266, respectively. The embodiment of the sound tests screen 246 illustrated in FIG. 41 identifies specific duration values that the user can select. It is noted that the specific duration values can change and the invention is not limited to the duration values illustrated in FIG. 41.

The A/D converter outputs selection 198 links to an A/D converter outputs screen 270 illustrated in FIG. 42. The A/D converter outputs screen 270 displays the data, e.g., EEG, ECG, and EMG signals, acquired from the patient 50. The A/D converter outputs screen 270 includes a data identification section 274 that identifies the channel from which the data has been acquired and the corresponding minimums and maximums of the displayed signal. A Begin button 276 commences acquisition and display of the data signal from the sensor or transducer. A Hold button 278 pauses the acquisition of the data signal from the sensor or transducer. During the pause period, the user can further examine the data signal and can adjust the parameters of the amplifier and filter circuit 102, which adjustments are affected in the paused data signal.

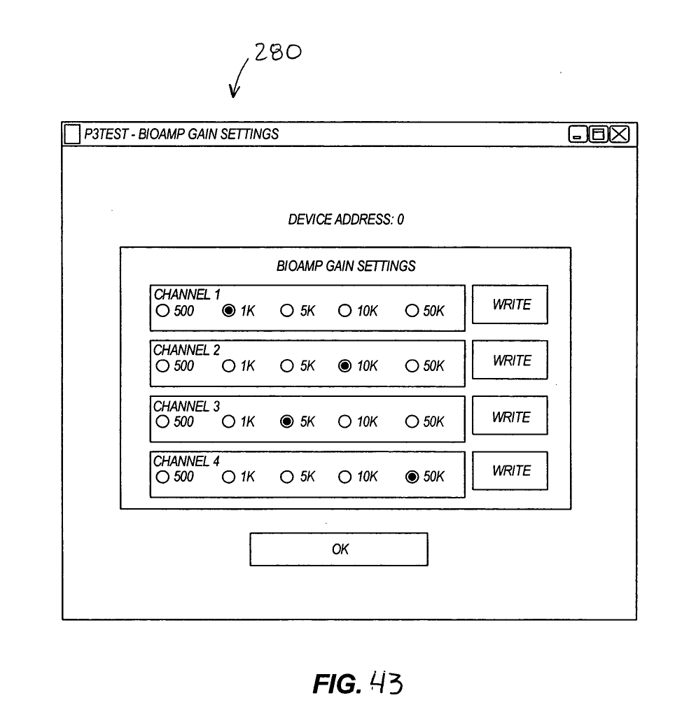

The bioamplifier gain settings selection 214 links to a bioamplifier gain settings screen 280 illustrated in FIG. 43. The user can select and/or adjust the amplifier gain of the amplifier and filter circuit 102 for each channel. The Write button initiates and saves the selections and/or adjustments to the amplifier and filter circuit 102. The embodiment of the bioamplifier gain settings screen 278 illustrated in FIG. 43 identifies specific gain values that the user can select. It is noted that the specific gain values can change and the invention is not limited to the gain values illustrated in FIG. 43.

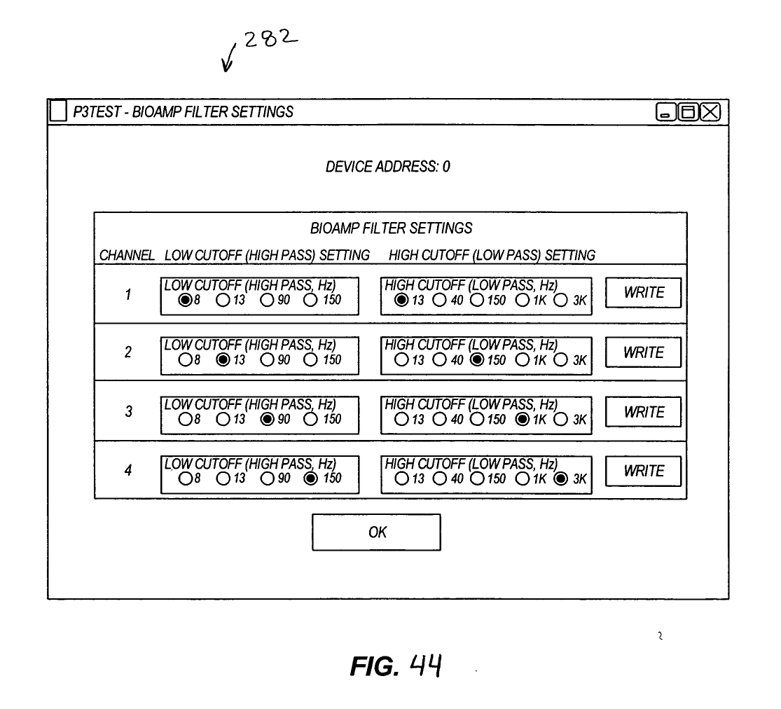

The bioamplifier filter settings selection 218 links to a bioamplifier filter settings screen 282 illustrated in FIG. 44. The user can select and/or adjust the low cutoff and high cutoff settings of the filters of the amplifier and filter circuit 102 for each channel. The Write button initiates and saves the selections and/or adjustments to the amplifier and filter circuit 102. The embodiment of the bioamplifier filter settings screen 282 illustrated in FIG. 44 identifies specific low cutoff and high cutoff values that the user can select. It is noted that the specific low cutoff and high cutoff values can change and the invention is not limited to the low cutoff and high cutoff values illustrated in FIG. 44.

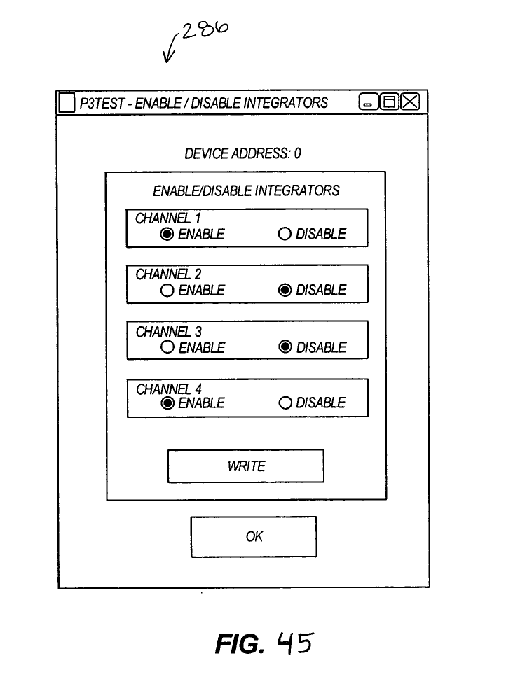

The enable/disable integrators selection 226 links to an enable/disable integrators screen 286 illustrated in FIG. 45. The user can select whether the integrator circuit 106 is enabled or disabled for each channel. The Write button initiates and saves the selection of whether the integrator circuit 106 is enabled or disabled for each channel.

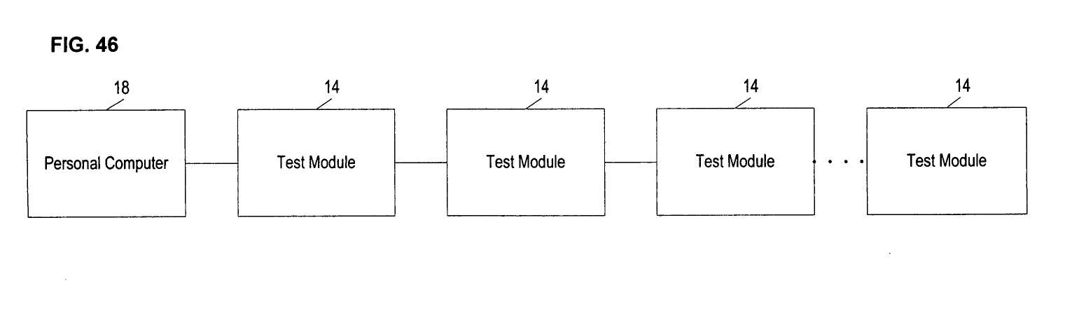

The personal computer 18 can accommodate data from a plurality of test modules 14. FIG. 46 illustrates a plurality of test modules 14 connected together according to one embodiment of the invention. Each of the test modules 14 includes the test module input 86 to connect to another test module 14. The software program 162 and user interface 182 can be modified to allow the user to select and/or adjust the parameters described above for the plurality of test modules 14. For example, the personal computer 18 can accommodate 10 test modules that are connected together, such that the user can interact with the user interface 182 and screens 242, 246, 270, 280, 282, and 286 to select and/or adjust the parameters for 40 amplifier and filter circuits 102, 10 skin conductance circuits 110, 40 channels of data 70, and 80 stimulus outputs 74.

The data acquired from the patient 50 can be stored in the storage device 166 of the personal computer 18 and/or the test module 14. Generally, each test conducted by the user is assigned a test number, which is stored in the personal computer 18 and associated with the acquired data from the patient 50. The test number can be any unique identifier, including alphanumerics, such as a patient identification number. The test number can be associated with additional patient information, such as name, age, weight, height, gender, etc., and test information, such as date of test, identification of user, location of test, etc.

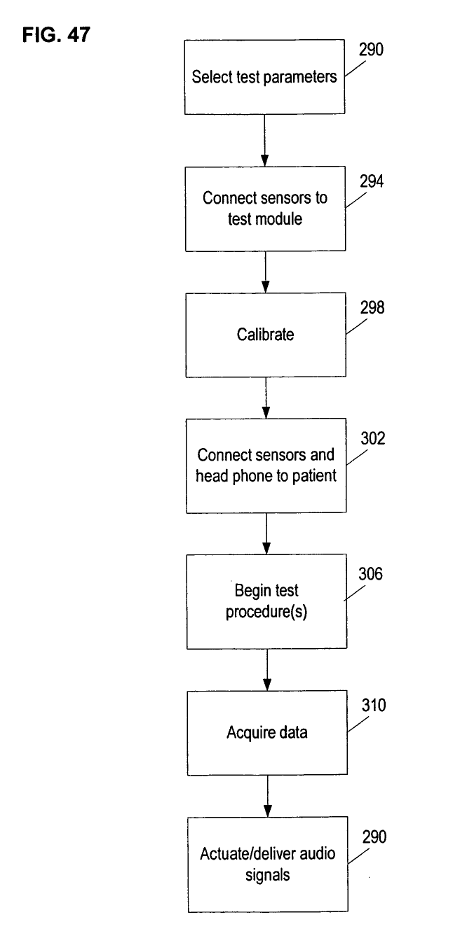

FIG. 47 illustrates a flow chart of an embodiment of the method of the invention. The user selects (at 290) the various test parameters to be administered to the patient 50. The user can select and/or adjust the amplifier gain, low cutoff and high cutoff filter settings, the particular stimuli to be administered, whether a tone signal and/or noise signal is to be administered and the corresponding amplitude, duration, and frequency, whether the integrator is enabled or disabled for each channel, and the skin conductance parameters. Additional parameters can also be selected and/or modified. The user connects (at 294) the sensors or transducers to the test module 14 via the input ports 42. The user calibrates (at 298) the sensors or transducers, if necessary. The user connects (at 302) the sensors or transducers and headphones (if applicable to the test) to the patient 50. The user begins (at 306) administering the test procedure(s) and the test module acquires (at 310) data from the patient 50. In some embodiments, the user instructs the software program 162 to begin acquiring data. During the test procedure(s), the user actuates (at 314) the audio signal generator circuit 114 to deliver selected stimuli, tones, and/or noise to the patient 50. The test module 14 and personal computer 18 continue to acquire data from the patient as the test(s) and audio signal(s) are delivered/administered to the patient 50. The user can repeat acts 290-314 any number of times for administering psychophysiological tests to the patient 50.

Various features and advantages of the invention are set forth in the following claims.

What is claimed is:

1. A biofeedback system comprising:
a test module including
an amplifier and filter circuit,
an integrator circuit,
a skin conductance circuit, and
an audio signal generator circuit operable to output one of a tone signal and a white noise signal; and
a processor including
a software program operable to adjust functionality of the amplifier and filter circuit, the integrator circuit, and the audio signal generator circuit,
an acquisition module operable to receive data from the test module, the data acquired from a patient, and
a display module operable to display the received data.

2. The biofeedback system of claim 1 wherein the software program is operable to measure a startle reflex of the patient.

3. The biofeedback system of claim 1 wherein the audio signal generator circuit is operable to output a plurality of stimuli different than the tone signal and the white noise signal.

4. The biofeedback system of claim 1 wherein the test module is operable to acquire a plurality of data signals from the patient.

5. The biofeedback system of claim 1 wherein the software program includes a user interface and a plurality of screens viewable by the user.

6. The biofeedback system of claim 5 wherein the user can browse the plurality of screens to select amplifier gain settings of the amplifier and filter circuit.

7. The biofeedback system of claim 5 wherein the user can browse the plurality of screens to select filter settings of the amplifier and filter circuit.

8. The biofeedback system of claim 5 wherein the user can browse the plurality of screens to select at least one of amplitude, duration, and frequency parameters of the tone signal generated by the audio signal generator circuit.

9. The biofeedback system of claim 5 wherein the user can browse the plurality of screens to select at least one of amplitude and duration parameters of the white noise signal generated by the audio signal generator circuit.

10. The biofeedback system of claim 1 wherein the data acquired from the patient is one of electrocardiogram data, electroencephalogram data, electromyogram data, and skin conductance data.

11. A biofeedback system comprising:
a plurality of test modules, each test module configured to link to another test module, each test module including
an amplifier and filter circuit, and
an integrator circuit; and
a processor connected to one of the test modules, the processor including
a software program operable to adjust functionality of the amplifier and filter circuit and the integrator circuit,
an acquisition module operable to receive data from the plurality of test modules, the data acquired from a patient, and
a display module operable to display the received data.

12. The biofeedback system of claim 11 wherein the test module further comprises a skin conductance circuit.

13. The biofeedback system of claim 11 wherein the test module further comprises an audio signal generator circuit operable to output one of a tone signal and a white noise signal.

14. The biofeedback system of claim 13 wherein the processor is operable to adjust the functionality of the audio signal generator circuit.

15. The biofeedback system of claim 11 wherein the test module further comprises a processor operable to provide a voltage signal to a stimuli delivery device.

16. The biofeedback system of claim 11 wherein the software program includes a user interface and a plurality of screens viewable by the user, and wherein the user can browse the plurality of screens to select amplifier gain settings and filter settings of the amplifier and filter circuit.

17. A method of acquiring psychophysiological data from a patient with a biofeedback system including a personal computer and a test module, the method comprising:
the test module receiving instructions from the personal computer to adjust functionality of the test module;
the test module delivering a plurality of stimuli to the patient;
the test module acquiring data from the patient, the data reflective of a response of the patient to the delivered stimuli;
transmitting the data to the personal computer; and
displaying the data on the personal computer.

18. The method of claim 17 wherein the act of the test module receiving instructions from the personal computer includes the act of receiving instructions related to adjusting functionality of one of an amplifier and filter circuit, an integrator circuit, and an audio signal generator circuit.

19. The method of claim 17 wherein the act of the test module acquiring data from the patient includes the act of acquiring at least one of electrocardiogram data, electroencephalogram data, electromyogram data, and skin conductance data.

20. The method of claim 17 further comprising the acts of receiving an instruction to pause acquisition of the data from the patient, receiving additional instructions from the personal computer to adjust functionality of the test module, and updating the paused patient data displayed on the personal computer.
